SEQUENCE LISTING (12 sequences)

SEQ ID NO: 1 - protein, 10 aa, Staphylococcus aureus
KKKVTAQELD

SEQ ID NO: 2 - protein, 31 aa, Staphylococcus aureus
RSITVRVFEDGKNLLSFDVQTNKKKVTAQEL

SEQ ID NO: 3 - protein, 10 aa, Staphylococcus aureus
KKNVTVQELD

SEQ ID NO: 4 - protein, 10 aa, Staphylococcus aureus
KKEVTVQELD

SEQ ID NO: 5 - protein, 10 aa, Staphylococcus aureus
KKQLAISTLD

SEQ ID NO: 6 - protein, 30 aa, Staphylococcus aureus
NKKKVTAQELDYLTRHYLVKNKKLYEFNNS

SEQ ID NO: 7 - DNA, 30 bp, Staphylococcus aureus
aagaaaaaggtgactgctcaagaattagat

SEQ ID NO: 8 - DNA, 93 bp, Staphylococcus aureus
agaagtattactgttcgggtatttgaagatggtaaaaatttattatcttttgacgtacaaactaataagaaaaaggtgactgctcaagaatta

SEQ ID NO: 9 - DNA, 90 bp, Staphylococcus aureus
attaagaaaaaggtgactgctcaagaattagattacctaactcgtcactatttggtgaaaaataaaaaactctatgaatttaacaactcg

SEQ ID NO: 10 - DNA, 30 bp, Staphylococcus aureus
aagaaaaatgtaactgttcaggagttggat

SEQ ID NO: 11 - DNA, 30 bp, Staphylococcus aureus
aaaaaagaagtaactgttcaagagctagat

SEQ ID NO: 12 - DNA, 30 bp, Staphylococcus aureus
aaaaaacaattagctatatcaactttagac

(19) United States
(12) Patent Application Publication
(10) Pub. No.: US 2005/0191315 A1
(43) Pub. Date: Sep. 1, 2005

(54) Identification of epitopes on staphylococcal enterotoxins

(76) Inventors: Carol Pontzer, Silver Spring, MD (US); Marti Jett, Washington, DC (US); Jeffrey Shupp, Hagerstown, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/903,478

(22) Filed: Aug. 2, 2004

Related U.S. Application Data
(63) Continuation-in-part of application No. PCT/US03/03139, filed on Feb. 3, 2003.
(60) Provisional application No. 60/353,365, filed on Feb. 1, 2002.

(51) Int. Cl. A61K 39/02; C07K 14/31

(52) U.S. Cl. 424/190.1; 530/350

(57) ABSTRACT

The present invention relates to novel peptides that inhibit transcytosis of Staphylococcal enterotoxins and methods for preventing and/or treating Staphylococcal enterotoxin-mediated diseases or conditions.

This application is a continuation-in-part of International Patent Application No. PCT/US03/03139, filed Feb. 3, 2003, which claims the benefit of U.S. Provisional Application No. 60/353,365 filed Feb. 1, 2002, which are hereby incorporated by reference in their entirety.

### FIELD OF THE INVENTION

The present invention relates to novel peptides that inhibit transcytosis of Staphylococcal enterotoxins. In particular, the present invention relates to novel Staphylococcal enterotoxin B peptides or epitopes that inhibit transcytosis of Staphylococcal enterotoxins. Further, the present invention provides methods for preventing and/or treating Staphylococcal enterotoxin-mediated diseases or conditions and methods for reducing transcytosis of a Staphylococcal enterotoxin thereby reducing systemic exposure to said enterotoxin.

The organism Staphylococcus aureus is a Gram-positive bacterium that is known for its production of potent toxins. Once introduced into a host's system these toxins can act to profoundly stimulate the immune system. The proteins are known to act on host systems in three distinct ways: as enterotoxins, they induce emesis and diarrhea in humans and non-human primates (Jett et al., Infect. Immun., 58:3494-3499 (1990)); as exotoxins, they have been implicated in the induction of toxic shock (Marrack et al., Science, 249:705-711 (1990)); and as superantigens, they induce extensive Vb-specific T cell stimulation (Herrmann et al., Eur. J. Immunol., 22:1935-1938 (1992)) followed by anergy and apoptosis which results in immunosuppression (Rellahan et al., J. Exp. Med., 172:1091-1100 (1990)).

S. aureus is carried by close to 30% of the world's population (Salyers et al., A. A., and D. D. Whitt. Disease without colonization: food-borne toxinoses caused by Clostridium botulinum, Staphylococcus aureus, and Clostridium perfringens. p. 136-139. In: Bacterial pathogenesis. ASM Press, Washington, D.C. (1994)). This high carrier rate may be a factor in the prevalence of S. aureus contamination of food. Poor hygiene and improper holding temperatures of meats and creamy dishes such as salad dressings have been implicated as the primary etiologies of food borne disease caused by S. aureus (Bean et al., Mor. Mortal. Wkly. Rep., 45:1-66 (1996)). At temperatures below 60° C., most strains of the bacteria produce enterotoxins. Once these enterotoxins enter the intestines of a host they have the ability to transcytose the intestinal wall and gain access to the immune system (Hamad et al., J. Exp. Med., 185:1447-1454 (1997); McKay et al., J. Immunol., 159:2382-2390 (1997)). Within 3-12 hours after aerosol exposure, symptoms such as high fever (103-106 degrees), chills, headache, myalgia, and nonproductive cough with or without shortness of breadth and chest pain can occur (Fever last approximately 2-5 days, coughs can persist for 4 weeks, and the individual is incapacitated for at least 2 weeks). Within four hours of ingestion, these toxins can be responsible for an array of symptoms such as: nausea, vomiting, abdominal pain, acute salivation and diarrhea with extensive fluid loss. Higher exposures (30 ng/kg) can lead to septic shock and death if untreated.

The host should become symptom free about 24 hours after the ingestion time. However, anorexia has been shown to persist for approximately 5 days in monkeys challenged with an incapacitation dose of SEB (M. Sipos and M. Jett, WRAIR-personal communication). The mechanism responsible for the emetic response to staphylococcal enterotoxins (SE) could be immune-mediated, in that stimulation of T cell proliferation is associated with massive cytokine production (Carlsson et al., Cell. Immunol., 96:175-183 (1985)). In fact, the symptoms of food poisoning can be mimicked by administration of exogenous interleukin 2 (IL-2) (Johnson et al., Sci Am., 266:92-101 (1992)). However, studies of SEA mutated at histidine 61 have shown that superantigenic and emetic activity can be separated (Hoffman et al., Infection and Immunity, 64:885-890 (1996)). Thus, additional mechanisms may be involved in elicitation of emesis. Further, drugs that block receptors for serotonin completely ablated the vomiting, diarrhea and prostration induced by SEA or SEB in piglets (Mani et al., submitted for publication).

Superantigens are able to stimulate unusually large numbers of T cells (up to 20% of the entire T cell complement of the host), and SE are prototypical bacterial superantigens. In contrast to conventional antigens, superantigens are not internalized by antigen-presenting cells, do not undergo processing, and are not presented within the antigen binding groove. Instead, the intact superantigens bind to both major histocompatibility complex (MHC) class II molecules and T cells at sites distinct from the conventional antigen-binding sites (Jardetzky et al., Nature, 368:188-192 (1994)). Indeed, it has been shown that interaction with superantigens primarily involves the variable region of the T cell receptor (TCR) b chain (Vb) (Johnson et al., Sci Am., 266:92-101 (1992)). Subsequent to proliferation, most T cells whose cognate antigen is not present in the host will undergo clonal deletion, resulting in immunosuppression. By contrast, in susceptible individuals, activated T cells may continue to be stimulated and precipitate autoimmune disease (Cole et al., ASM News, 62:471-475 (1996); Johnson et al., Sci Am., 266:92-101 (1992)).

The biological significance of superantigenic SE as causative agents of food poisoning and toxic shock contribute to the importance of this invention (Jett et al., Infect Immun., 58:3494-9 (1990); Marrack et al., Science, 249:705-11 (1990)). Toxic shock caused by SE can follow wound infections, staphylococcal co-infections with influenza, and could result from toxins used as agents of biological warfare. The hazards resulting from these interactions include not just toxic shock, but sequelae such as precipitation and exacerbation of autoimmune disease in susceptible members of the population (Cole et al., ASM News, 62:471-5 (1996), Johnson et al., Sci Am., 266:92-101 (1992)). The paradoxical immune suppression, which normally follows T cell stimulation, may be particularly devastating for individuals with impaired immune function such as AIDS patients. Increased risk for both of these sequelae may occur at exposure levels below those required for emetic and toxic shock responses.

The area of the toxins identified as involved in transcytosis is remarkably structurally conserved within the SEs, and may present the appropriate surface conformation to the transcytosis receptor. The structural interactions of SE with both MHC and TCR have been well defined (Jardetzky et al., Nature, 368:188-92 (1994); Malchiodi et al., J Exp Med., 182:1833-45 (1995)). Mutations at SEB residue N23 that interacts with the TCR and F44 that binds MHC II have been associated with reduced toxin transcytosis by Caco-2 cells (Hamad, et al., J. Exp. Med., 185:1447-54 (1997)). However, the ability of SE to stimulate T cells via MHC:TCR interaction cannot be directly correlated with their emetic activity (Harris et al., Infect. Immun., 61:3175-83 (1993)), suggesting that alternative regions of the molecule may be involved in toxin activity in the gut. The sequence KKKVTAQEL in SEB has been recognized previously as being highly conserved among the SE, and it has been suggested that antisera directed against this region may neutralize toxin activity (Jett et al., Infect Immun., 62:3408-15 (1994)). Further, the longer SEB 130-160 peptide conjugated to KLH decreased SEB binding to lymphocytes (Jett et al., Infect Immun., 62:3408-15 (1994)). Recently, a variant of an SEB peptide representing amino acids 150-161 with the alanine and valine inverted and tyrosine replacing threonine 150 was found to protect mice against intraperitoneally SEB-induced lethal shock (Arad et al., Nature Med., 6:414-21 (2000)). The protective effect was observed when the peptide was injected intravenously 30 minutes before toxin or even three hours after toxin administration. This time frame of activity is consistent with our observation that SEB 152-161 inhibits transcytosis, and the transcytosis time course experiments showed no significant toxin movement till well beyond the three hour point. A/J and Balb/c mice were both protected by the peptide (Arad et al., Nature Med., 6:414-21 (2000)). This suggests that the transcytosis receptor is distinct from class II MHC molecules.

### DESCRIPTION OF THE INVENTION

Staphylococcal enterotoxins are exoproteins produced by S. aureus that act as superantigens and have been implicated as a leading cause of food borne disease and toxic shock. To model movement in vitro of staphylococcal enterotoxins, a monolayer system composed of crypt-like human colonic T-84 cells has been employed. SEB and SEA showed comparable dose dependent transcytosis in vitro, while TSST-1 exhibited increased movement at lower doses. Synthetic peptides corresponding to specific regions of the SEB molecule were tested in vitro to identify the domain of the protein involved in the transcytosis of SE. A toxin peptide of particular interest contains the amino acid sequence KKKVTAQELD, that is highly conserved across all SE. At a toxin:peptide ratio of 1:10, movement of SEB across the monolayers was reduced by 85%. Antisera made against the SEB peptide recognized native SEB and also inhibited SEB transcytosis. Finally, the conserved 10 amino acid peptide inhibited transcytosis of multiple staphylococcal enterotoxins, SEA, SEE and TSST-1. These data demonstrate that this region of the staphylococcal enterotoxins plays a distinct role in toxin movement across epithelial cells. It has implications for the prevention of staphylococcal enterotoxin-mediated disease by design of a peptide vaccine that could reduce systemic exposure to oral or inhaled superantigens. Since the sequence identified is highly conserved, it allows for a single epitope blocking the transcytosis of multiple SE.

Thus, the present invention relates to novel peptides (SEQ ID NOs:1-6) that inhibit transcytosis of Staphylococcal enterotoxins. The present invention also relates to novel Staphylococcal enterotoxin B peptides (SEQ ID NOs:1-2 and 6) that inhibit transcytosis of Staphylococcal enterotoxins.

Still further, the present invention provides methods for preventing and/or treating Staphylococcal enterotoxin-mediated diseases or conditions and methods for reducing transcytosis of a Staphylococcal enterotoxin thereby reducing systemic exposure to said enterotoxin.

One aspect of the present invention is to provide an isolated polypeptide as represented by a sequence selected from the group consisting of SEQ ID NO:1, SEQ ID NO:2, SEQ ID NO:3, SEQ ID NO:4, SEQ ID NO:5 and SEQ ID NO:6, or a fragment or variant thereof. As a preferred embodiment, the present invention provides an isolated polypeptide represented by SEQ ID NO:1 (KKKVTAQELD) from Staphylococcal enterotoxin B.

Another aspect of the present invention is to provide an isolated polynucleotide as represented by a sequence selected from the group consisting of SEQ ID NO:7, SEQ ID NO:8, SEQ ID NO:9, SEQ ID NO:10, SEQ ID NO:11 and SEQ ID NO:12, or a fragment or variant thereof. Further, the present invention provides an isolated polynucleotide, or a fragment or variant thereof, which encodes a polypeptide of SEQ ID NOS:1-6. A typical polynucleotide is the sequence of SEQ ID NO:7.

Another aspect of the invention is an isolated polynucleotide which comprises a nucleotide sequence that codes without interruption for the polypeptides of SEQ ID NOs:1-5 or 6, or a fragment or variant of SEQ ID NOs:1-5 or 6, or that is the complement of a sequence that codes without interruption for the polypeptide of SEQ ID NOs:1-5 or 6 or a fragment or variant thereof. A polynucleotide, which “codes without interruption” refers to a polynucleotide having a continuous open reading frame (“ORF”) as compared to an ORF, which is interrupted by introns or other noncoding sequences.

The invention also relates to methods of making the above-described polypeptides or polynucleotides (e.g., methods of making constructs which comprise and/or express the polynucleotide sequences; and methods of transforming cells with constructs capable of expressing the polypeptides, culturing the transformed cells under conditions effective to express the polypeptides, and harvesting (recovering) the polypeptides); to antibodies, antigen-specific fragments, or other specific binding partners which are specific (selective) for the polypeptides; to methods of using polypeptides, polynucleotides or antibodies of the invention to detect the presence or absence, and/or to quantitate the amounts, of the polypeptides and polynucleotides of the invention in a sample; to transgenic animals which express the polypeptides or for other potential uses.

For example, the invention relates to an isolated polypeptide comprising the amino acid sequence of SEQ ID NO:1-5 or 6, or a fragment or variant of SEQ ID NO:1-5 or 6. The polypeptide may comprise, e.g., at least about 40%, 45%, 50%, 55%, 60%, 65%, 70%, 75%, 80%, 85%, 90%, 95%, 97% or 99% sequence identity to SEQ ID NO:1-5 or 6 or a fragment thereof; and/or may comprise a sequence that is substantially homologous to SEQ ID NO:1-5 or 6 or a fragment thereof.

In another aspect, the invention relates to an isolated polynucleotide which comprises the nucleotide sequence of SEQ ID NO:7-11 or 12 or a fragment or variant of SEQ ID NO: 7-11 or 12 or a complement thereof. The polynucleotide may further be DNA, cDNA, RNA, PNA or combinations thereof. The polynucleotide may comprise a sequence that hybridizes to SEQ ID NO: 7-11 or 12 or a fragment thereof under conditions of high stringency; and/or may comprise a sequence that is substantially homologous to SEQ ID NO: 7-11 or 12 or a fragment thereof; and/or may have a sequence identity of, e.g., at least about 40%, 45%, 50%, 55%, 60%, 65%, 70%, 75%, 80%, 85%, 90%, 95%, 97% or 99% to SEQ ID NO: 7-11 or 12 or a fragment thereof.

In another aspect, the invention relates to a recombinant construct comprising a polynucleotide as above, which may be operatively linked to a regulatory sequence, e.g., wherein said construct comprises an expression vector. The invention also relates to a cell comprising such a construct, e.g., a prokaryotic, mammalian, human, yeast or insect cell. The invention also relates to a method of making such a cell, comprising introducing a construct or polynucleotide as above into a cell. The invention also relates to a method to make a polypeptide of the invention, comprising incubating a cell as above under conditions in which the polypeptide is expressed, and harvesting the polypeptide.

Another aspect, the invention relates to a pharmaceutical composition comprising a polypeptide or polynucleotide of the invention and a pharmaceutically acceptable carrier. In another aspect, the invention relates to a prophylactic or therapeutic method of treating a disease or condition mediated by staphylococcal enterotoxin, comprising administering to a patient in need thereof a therapeutically effective amount of said composition.

In another aspect, the invention relates to an antibody, antigen-specific antibody fragment, or other specific binding partner, which is specific for a polypeptide of the invention, e.g., wherein said antibody, antigen-specific antibody fragment, or specific binding partner is specific for the polypeptide of SEQ ID NO:1-5 or 6. The invention also relates to a pharmaceutical composition comprising an antibody and a pharmaceutically acceptable carrier.

In another aspect, the polypeptides or polynucleotides of the invention can be administered to a host as a vaccine.

In another aspect, the invention relates to a method for preventing or treating a staphylococcal enterotoxin-mediated disease or condition comprising administering a therapeutically effective amount of a polypeptide, an antibody, or a composition as described above. The staphylococcal enterotoxin-mediated disease or condition can be, for example, emesis, diarrhea, toxic shock, or immunosuppression.

In yet another aspect, the invention relates to a method for reducing transcytosis of and reducing systemic exposure to a staphylococcal enterotoxin comprising administering a therapeutically effective amount of a polypeptide, an antibody, or a composition as described above. The staphylococcal enterotoxin can be, for example, staphylococcal enterotoxin A-J or TSST-1.

In another aspect, the invention relates to a transgenic animal (e.g., a mouse) comprising at least one copy of a polynucleotide of the invention, wherein the animal overexpresses the polynucleotide, or a functional fragment or analog thereof, compared to a non-transgenic animal.

The present invention also relates to methods for testing agents for treating and/or preventing a staphylococcal enterotoxin-mediated disease or condition, comprising, e.g., administering an agent for treating and/or preventing a staphylococcal enterotoxin-mediated disease or condition to a piglet, wherein the piglet has been administered an amount of SEB which is effective for causing a staphylococcal enterotoxin-mediated disease or condition, and determining whether said agent is effective for treating and/or preventing a staphylococcal enterotoxin-mediated disease or condition. Example VI below provides an example of an animal model using piglets. An agent can be any compound, drug, or material that is to be tested for its efficacy in treating and/or preventing a staphylococcal enterotoxin-mediated disease or condition.

Polypeptides

S. aureus produces multiple serotypes of toxin; staphylococcal enterotoxin A (SEA), SEB, SEC1, SEC2, SEC3, SED, SEE through SEJ and toxic shock syndrome toxin one (TSST-1) (Jarraud et al., J. Immunol., 166:669-677 (2001)). SEB and SEA are the most clinically important and best characterized bacterial superantigenic toxins. SEB is a single polypeptide chain of approximately 28.5 kDa. The protein structure consists of 5 α-helices and 12 β-sheets organized into two domains (Swaminathan et al., Nature, 359:801-806 (1992)).

Subsequent to resolution of the SEB crystal structure, attempts have been made to identify the regions of the toxins important for their immunomodulatory effects. SE have been co-crystallized with either class II MHC or the TCR region to which they bind (Jardetzky et al., Nature, 368:188-192 (1994); Malchiodi et al., J Exp Med., 182:1833-1845 (1995)). The important regions determined by resolution of these crystal structures largely confirm the data obtained functionally from peptides and mutant toxins. The MHC-binding region of SEB consists of residues in helix 5 and β-sheet 2, while the TCR-binding regions include four discrete sequences at the top of the molecule, helix 2, the β-sheet 2-3 loop, the end of β-sheet 4 and the β10 to helix 5 loop. Thus, the sites of interaction of SE with immune cells have been well documented. In contrast, studies to determine the region of the SE involved in their entry into the body through gastrointestinal or pulmonary surfaces in order to gain access to local and systemic immune systems have been much more limited. The present invention identifies a region of SEB distinct from MHC or TCR binding sites that is involved in transcytosis across epithelial cell monolayers. Further, a peptide corresponding to amino acids 152-161 within this region of SEB significantly inhibits transcytosis of not just SEB, but transcytosis of multiple other SEs as well.

A polypeptide of the present invention may be a recombinant polypeptide, a natural polypeptide or a synthetic or semi-synthetic polypeptide, or combinations thereof, preferably a recombinant or synthesized polypeptide. As used herein, the terms polypeptide, oligopeptide and protein are interchangeable.

The polypeptides of the present invention are preferably provided in an isolated form, and may be purified, e.g., to homogeneity. The term “isolated,” when referring, e.g., to a polypeptide or polynucleotide, means that the material is removed from its original environment (e.g., the natural environment if it is naturally occurring), and isolated or separated from at least one other component with which it is naturally associated. For example, a naturally-occurring polypeptide present in its natural living host is not isolated, but the same polypeptide, separated from some or all of the coexisting materials in the natural system, is isolated. Such polypeptides could be part of a composition, and still be isolated in that such composition is not part of its natural environment.

The terms “fragment” or “variant,” when referring to a polypeptide of the invention mean a polypeptide which retains substantially at least one of the biological functions or activities of the polypeptide. Such a biological function or activity can be, e.g., any of those described above, and includes having the ability to react with an antibody, i.e., having an epitope-bearing peptide. Fragments or variants of the polypeptides have sufficient similarity to those polypeptides so that at least one activity of the native polypeptides is retained. Fragments or variants of smaller polypeptides retain at least one activity (e.g., the ability to react with an antibody or antigen-binding fragment of the invention) of a comparable sequence found in the native polypeptide. In one embodiment, a fragment is at least 10 amino acids of an polypeptide sequence or a polynucleotide sequence that encodes for at least 10 amino acids.

Polypeptide fragments of the invention may be of any size that is compatible with the objects of the invention. Fragments of the polypeptides of the present invention may be employed, e.g., for producing the corresponding full-length polypeptide by peptide synthesis, e.g., as intermediates for producing the full-length polypeptides; for inducing the production of antibodies or antigen-binding fragments; as “query sequences” for the probing of public databases, or the like.

A variant of a polypeptide of the invention may be, e.g., (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which additional amino acids are fused to the polypeptide, such as a leader or secretory sequence or a sequence which is employed for purification of the polypeptide, commonly for the purpose of creating a genetically engineered form of the protein that is susceptible to secretion from a cell, such as a transformed cell. The additional amino acids may be from a heterologous source, or may be endogenous to the natural gene.

Variant polypeptides belonging to type (i) above include, e.g., muteins, analogs and derivatives. A variant polypeptide can differ in amino acid sequence by, e.g., one or more additions, substitutions, deletions, insertions, inversions, fusions, and truncations or a combination of any of these.

Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins—Structure and Molecular Properties, 2nd ed., T.E. Creighton, W.H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., Meth. Enzymol., 182:626-46 (1990) and Rattan et al., Ann. N.Y. Acad. Sci., 663:48-62 (1992).

Variant polypeptides belonging to type (iii) are well known in the art and include, e.g., PEGulation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical examples of such variants are discussed elsewhere herein.

Many other types of variants are known to those of skill in the art. For example, as is well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally occurring and synthetic polypeptides. For instance, the amino terminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

As noted above, the polypeptides of the present invention include, e.g., isolated polypeptides comprising the sequence of SEQ ID NO:1-5 or 6 and fragments thereof. The polypeptides of the invention also include polypeptides which have varying degrees of sequence homology (identity) thereto or that shows substantial sequence homology (sequence identity).

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100 [1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence.

The description herein for percent identity or percent homology is intended to apply equally to nucleotide or amino acid sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:3389-3402 (1997). When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (J. Mol. Biol., 48:444-453 (1970)) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5 or 6. In yet another preferred embodiment, the percent identity between two nucleotide sequences is determined using the GAP program I the GCG software package (Devereux et al., Nucleic Acids Res., 12 (1):387 (1984)) using a NWSgapdna. CMP matrix and a gap weight of 40, 50, 60, 70, or 80 and a length weight of 1, 2, 3, 4, 5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al., Comput. Appl. Biosci., 10:3-5 (1994); and FASTA described in Pearson et al., Proc. Natl. Acad. Sci. USA, 85:2444-8 (1988).

In accordance with the present invention, the term “substantially homologous,” when referring to a protein sequence, means that the amino acid sequences are at least about 90-95% preferably 97-99% or more identical. A substantially homologous amino acid sequence of the invention can be encoded by a nucleic acid sequence hybridizing to the nucleic acid sequence, or portion thereof, of the sequence shown in SEQ ID NOs:7-12, under conditions of high stringency.

Conditions of “high stringency,” as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

As used with respect to the polypeptides (and polynucleotides) of the present invention, the term fragment refers to a sequence that is a subset of a larger sequence (i.e., a continuous or unbroken sequence of residues within a larger sequence).

Nucleic Acids

As discussed above, the invention includes polynucleotides, e.g., cDNAs, encoding the polypeptides of the invention, and fragments thereof.

The polynucleotide sequence of SEQ ID NOs:7-12 contains an open reading frame available for the coding of polypeptide amino acid sequence.

As used herein, the phrase “an isolated polynucleotide which is SEQ ID NO,” or “an isolated polynucleotide which is selected from SEQ ID NO,” includes an isolated nucleic acid molecule from which the recited sequence was obtained (i.e., the mRNA). Because of sequencing errors, typographical errors, etc., the actual naturally occurring sequence may differ from a SEQ ID listed herein. Thus, the phrase includes the specific molecule from which the sequence was derived, rather than a molecule having that exact recited nucleotide sequence, analogously to how a culture depository number refers to a specific cloned fragment in a cryotube.

A polynucleotide of the present invention may be a recombinant polynucleotide, a natural polynucleotide, or a synthetic or semi-synthetic polynucleotide, or combinations thereof. As used herein, the terms polynucleotide, oligonucleotide, oligomer and nucleic acid are interchangeable.

As used herein, the term “gene” means a segment of DNA involved in producing a polypeptide chain; it may include regions preceding and following the coding region (leader and trailer) as well as intervening sequences (introns) between individual coding segments (exons). Of course, cDNAs lack the corresponding introns. The invention includes isolated genes (e.g., genomic clones) which encode polypeptides of the invention.

Polynucleotides of the invention may be RNA, PNA, or DNA, e.g., cDNA, genomic DNA, and synthetic or semi-synthetic DNA, or combinations thereof. The DNA may be triplex, double-stranded or single-stranded, and if single stranded, may be the coding strand or non-coding (anti-sense) strand. It can comprise hairpins or other secondary structures. The RNA includes oligomers (including those having sense or antisense strands), mRNAs, polyadenylated RNA, total RNA, single strand or double strand RNA, or the like. DNA/RNA duplexes are also encompassed by the invention.

The polynucleotides and fragments thereof of the present invention may be of any size that is compatible with the invention, e.g., of any desired size that is effective to achieve a desired specificity when used as a probe. Polynucleotides may range in size, e.g., from the smallest specific probe (e.g., about 10-12 nucleotides) to greater than a full-length DNA, e.g., in the case of a fusion polynucleotide or a polynucleotide that is part of a genomic sequence; fragments may be as large as, e.g., one nucleotide shorter than a full-length cDNA.

A fragment of a polynucleotide according to the invention may be used, e.g., as a hybridization probe, as discussed elsewhere herein.

Many types of variants of polynucleotides are encompassed by the invention including, e.g., (i) one in which one or more of the nucleotides is substituted with another nucleotide, or which is otherwise mutated; or (ii) one in which one or more of the nucleotides is modified, e.g., includes a subtituent group; or (iii) one in which the polynucleotide is fused with another compound, such as a compound to increase the half-life of the polynucleotide; or (iv) one in which additional nucleotides are covalently bound to the polynucleotide, such a sequences encoding a leader or secretory sequence or a sequence which is employed for purification of the polypeptide. The additional nucleotides may be from a heterologous source, or may be endogenous to the natural gene.

Polynucleotide variants belonging to type (i) above include, e.g., polymorphisms, including single nucleotide polymorphisms (SNPs), degenerate and allelic variants and mutants. Variant polynucleotides can comprise, e.g., one or more additions, insertions, deletions, substitutions, transitions, transversions, inversions, chromosomal translocations, variants resulting from alternative splicing events, or the like, or any combinations thereof.

A coding sequence which encodes a polypeptide of the invention may be identical to the coding sequence shown in SEQ ID NO:7-11 or 12 or a fragment thereof, or may be a different coding sequence, which coding sequence, as a result of the redundancy or degeneracy of the genetic code, encodes the same polypeptide as the DNA of SEQ ID NO:7-11 or 1.2 or a fragment thereof. Such a peptide is sometimes referred to herein as a “degenerate variant.” Alternatively, the coding sequence may encode a polypeptide that is substantially homologous to the polypeptide of SEQ ID NO:1-5 or 6 or a fragment thereof.

A polynucleotide of the invention may have a coding sequence which is a naturally or non-naturally occurring allelic variant of a coding sequence encompassed by the sequence in SEQ ID NO:7-11 or 12. As known in the art, an allelic variant is an alternate form of a polynucleotide sequence, which may have a substitution, deletion or addition of one or more nucleotides, which in general does not substantially alter the function of the encoded polypeptide.

Other variant sequences, located in a coding sequence or in a regulatory sequence, may affect (enhance or decrease) the production of, or the function or activity of, a polypeptide of the invention.

Polynucleotide variants belonging to type (ii) above include, e.g., modifications such as the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; and 5,478,893.

Polynucleotide variants belonging to type (iii) above are well known in the art and include, e.g., various lengths of polyA⁺ tail, 5′cap structures, and nucleotide analogs, e.g., inosine, thionucleotides, or the like.

Polynucleotide variants belonging to type (iv) above include, e.g., a variety of chimeric, hybrid or fusion polynucleotides. For example, a polynucleotide of the invention can comprise a coding sequence and additional non-naturally occurring or heterologous coding sequence (e.g., sequences coding for leader, signal, secretory, targeting, enzymatic, fluorescent, antibiotic resistance, and other functional or diagnostic peptides); or a coding sequence and non-coding sequences, e.g., untranslated sequences at either a 5′ or 3′ end, or dispersed in the coding sequence, e.g., introns.

More specifically, the present invention includes polynucleotides wherein the coding sequence for the polypeptide (e.g., a mature polypeptide) is fused in the same reading frame to a polynucleotide sequence (e.g., a heterologous sequence), e.g. one which aids in expression and secretion of a polypeptide from a host cell, for example, a leader sequence which functions as a secretory sequence for controlling transport of a polypeptide from the cell and/or a transmembrane anchor which facilitates attachment of the polypeptide to a cellular membrane. A polypeptide having a leader sequence is a preprotein and may have the leader sequence cleaved by the host cell to form a mature form of the polypeptide. The polynucleotides may also encode for a proprotein which is the mature protein plus additional N-terminal amino acid residues. A mature protein having a prosequence is a proprotein and is generally an inactive form of the protein. Once the prosequence is cleaved an active protein remains.

Polynucleotides of the present invention may also have a coding sequence fused in frame to a marker sequence that allows for identification and/or purification of the polypeptide of the present invention. The marker sequence may be, e.g., a hexa-histidine tag (e.g., as supplied by a pQE-9 vector) to provide for purification of the mature polypeptide fused to the marker in the case of a bacterial host, or, for example, the marker sequence may be a hemagglutinin (HA) tag when a mammalian host is used. The HA tag corresponds to an epitope derived from the influenza hemagglutinin protein (Wilson et al., Cell, 37:767 (1984)).

Other types of polynucleotide variants will be evident to one of skill in the art. For example, the nucleotides of a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Also, polynucleotides of the invention may have a coding sequence derived from another genetic locus of an organism, providing it has a substantial homology to, e.g., part or all of the sequence of SEQ ID NO:7-11 or 12 or from another organism (e.g., an ortholog).

Of course, it is understood that variants exclude any sequences disclosed prior to the invention.

Polynucleotides according to the present invention can be labeled according to any desired method. The polynucleotide can be labeled using radioactive tracers such as, e.g., ³²P, ³⁵S, ³H, or ¹⁴C. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

The present invention includes polynucleotides encoding all of the polypeptides and fragments or variants thereof, as disclosed hereinabove. For example, a polynucleotide of the invention may comprise a sequence which has a sequence identity of at least about 65-100%, (e.g., at least about 70-75%, 80-85%, 90-95% preferably 97-99%) to, or which is substantially homologous to, or which hybridizes under conditions of high stringency to, the nucleotide sequence of SEQ ID NO:7-11 or 12, or to a fragment thereof; or which is complementary to one of those sequences.

The term “substantially homologous,” when referring to polynucleotide sequences, means that the nucleotide sequences are at least about 90-95% or 97-99% or more identical.

Constructs

The present invention also relates to recombinant constructs that contain vectors plus polynucleotides of the present invention. Such constructs comprise a vector, such as a plasmid or viral vector, into which a polynucleotide sequence of the invention has been inserted, in a forward or reverse orientation.

Large numbers of suitable vectors are known to those of skill in the art, and many are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as it is replicable and viable in the host.

In a preferred embodiment, the vector is an expression vector, into which a polynucleotide sequence of the invention is inserted so as to be operatively linked to an appropriate expression control (regulatory) sequence(s) (e.g., promoters and/or enhancers) which directs mRNA synthesis. Appropriate expression control sequences, e.g., regulatable promoter or regulatory sequences known to control expression of genes in prokaryotic or eukaryotic cells or their viruses, can be selected for expression in prokaryotes (e.g., bacteria), yeast, plants, mammalian cells or other cells. Preferred expression control sequences are derived from highly-expressed genes, e.g., from operons encoding glycolytic enzymes such as 3-phosphoglycerate kinase (PGK), α-factor, acid phosphatase, or heat shock proteins, among others. Such expression control sequences can be selected from any desired gene, e.g using CAT (chloramphenicol transferase) vectors or other vectors with selectable markers. Two appropriate vectors for such selection are pKK232-8 and pCM7.

Particular named bacterial promoters which can be used include lacI, lacZ, T3, T7, gpt, lambda P_{R}, P_{L} and trp. Eukaryotic promoters include CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the DNA encoding the polypeptides of the present invention by higher eukaryotes can be increased by inserting an enhancer sequence into the expression vector. Enhancers are cis-acting elements of DNA, usually about from 10 to 300 bp that act on a promoter to increase its transcription. Representative examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors also include origins of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Large numbers of suitable expression vectors are known to those of skill in the art, and many are commercially available. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, adeno-associated virus, TMV, fowl pox virus, and pseudorabies. However, any other vector may be used as long as it is replicable and viable in a host. Appropriate cloning and expression vectors for use with prokaryotic and eukaryotic hosts are described, e.g., by Sambrook, et al., Molecular Cloning: A Laboratory Manual, Second Edition, Cold Spring Harbor, N.Y., (1989), Wu et al., Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al., Eds.,), John Wiley & Sons, NY (1994-1999).

In a preferred embodiment, a Baculovirus-based expression system is used. Baculoviruses represent a large family of DNA viruses that infect mostly insects. The prototype is the nuclear polyhedrosis virus (AcMNPV) from Autographa californica, which infects a number of lepidopteran species. One advantage of the baculovirus system is that recombinant baculoviruses can be produced in vivo. Following co-transfection with transfer plasmid, most progeny tend to be wild type and a good deal of the subsequent processing involves screening. To help identify plaques, special systems are available that utilize deletion mutants. By way of non-limiting example, a recombinant AcMNPV derivative (called BacPAK6) has been reported in the literature that includes target sites for the restriction nuclease Bsu36I upstream of the polyhedrin gene (and within ORF 1629) that encodes a capsid gene (essential for virus viability). Bsf36I does not cut elsewhere in the genome and digestion of the BacPAK6 deletes a portion of the ORF1629, thereby rendering the virus non-viable. Thus, with a protocol involving a system like Bsu36I-cut BacPAK6 DNA most of the progeny are non-viable so that the only progeny obtained after co-transfection of transfer plasmid and digested BacPAK6 is the recombinant because the transfer plasmid, containing the exogenous DNA, is inserted at the Bsu36I site thereby rendering the recombinants resistant to the enzyme. See Kitts and Possee, A method for producing baculovirus expression vectors at high frequency, BioTechniques, 14, 810-817 (1993). For general procedures, see King and Possee, The Baculovirus Expression System: A Laboratory Guide, Chapman and Hall, New York (1992) and Recombinant Gene Expression Protocols, in Methods in Molecular Biology, Vol. 62, (Tuan, ed., Humana Press, Totowa, N.J., 1997), at Chapter 19, pp. 235-246.

Appropriate DNA sequences may be inserted into a vector by any of a variety of procedures. In general, the DNA sequence is inserted into an appropriate restriction endonuclease site(s) by procedures known in the art. Such procedures and others are deemed to be within the scope of those skilled in the art. Conventional procedures for this and other molecular biology techniques discussed herein are found in many readily available sources, e.g., Sambrook, et al., Molecular Cloning: A Laboratory Manual, Second Edition, Cold Spring Harbor, N.Y., (1989). If desired, a heterologous structural sequence is assembled in an expression vector in appropriate phase with translation initiation and termination sequences, and preferably, a leader sequence capable of directing secretion of translated protein into the periplasmic space or extracellular medium.

Transformed Cells and Methods of Producing Polypeptides of the Invention

The present invention also relates to host cells which are transformed/transfected/transduced with constructs such as those described above, and to progeny of said cells, especially where such cells result in a stable cell line that can be used for the production (e.g., preparative production) of the polypeptides of the invention.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9 (and other insect expression systems); animal cells, including mammalian cells such as CHO, QM7, COS (e.g., the COS-7 lines of monkey kidney fibroblasts described by Gluzman, Cell, 23:175 (1981)), C127, 3T3, CHO, HeLa, BHK or Bowes melanoma cell lines; plant cells, etc. The selection of an appropriate host is deemed to be within the knowledge of those skilled in the art based on the teachings herein.

Introduction of a construct into a host cell can be effected by, e.g., calcium phosphate transfection, DEAE-Dextran mediated transfection, lipofection a gene gun, or electroporation (Davis, L., Dibner, M., Battey, I., Basic Methods in Molecular Biology, (1986)).

Following transformation of a suitable host strain and growth of the host strain to an appropriate cell density, the selected promoter can be induced by appropriate means (e.g., temperature shift or chemical induction) if desired, and cells cultured for an additional period. The engineered host cells are cultured in conventional nutrient media modified as appropriate for activating promoters (if desired), selecting transformants or amplifying the genes of the present invention. The culture conditions, such as temperature, pH and the like, are those previously used with the host cell selected for expression, and will be apparent to the ordinarily skilled artisan.

Cells are typically harvested by centrifugation, disrupted by physical or chemical means, and the resulting crude extract retained for further purification. Alternatively, when a heterologous polypeptide is secreted from the host cell into the culture fluid, supernatants of the culture fluid can be used as a source of the protein. Microbial cells employed in expression of proteins can be disrupted by any convenient method, including freeze-thaw cycling, sonication, mechanical disruption, or use of cell lysing agents, such methods being well known to those skilled in the art.

The polypeptide can be recovered and purified from recombinant cell cultures by conventional methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or the like. Protein refolding steps can be used, as necessary, in completing configuration of the mature protein. High performance liquid chromatography (HPLC) can be employed for final purification steps.

In addition to the methods described above for producing polypeptides recombinantly from a prokaryotic or eukaryotic host, polypeptides of the invention can be prepared from natural sources, or can be prepared by chemical synthetic procedures (e.g., synthetic or semi-synthetic), e.g., with conventional peptide synthesizers. Cell-free translation systems can also be employed to produce such proteins using RNAs derived from the DNA constructs of the present invention. Proteins of the invention can also be expressed in, and isolated and/or purified from, transgenic animals or plants. Procedures to make and use such transgenic organisms are conventional in the art. Some such procedures are described elsewhere herein.

Antibodies, Antigen-Binding Fragments or Other Specific Binding Partners

The polypeptides, their fragments or variants thereof, or cells expressing them can also be used as immunogens to produce specific antibodies, or antigen-binding fragments, thereto. By a “specific” antibody or antigen-binding fragment is meant one, which binds selectively (preferentially) to a polypeptide of the invention, or to a fragment or variant thereof. An antibody “specific” for a polypeptide means that the antibody recognizes a defined sequence of amino acids within or including the polypeptide.

Antibodies of the invention can be, for example, polyclonal or monoclonal antibodies. The present invention also includes chimeric, recombinant, single chain, and partially or fully humanized antibodies, as well as Fab fragments, or the product of a Fab expression library, and fragments thereof. The antibodies can be IgM, IgG, subtypes, IgG2A, IgG1, etc. Various procedures known in the art may be used for the production of such antibodies and fragments.

Antibodies generated against the polypeptides corresponding to a sequence of the present invention can be obtained, e.g., by direct injection of the polypeptides into an animal or by administering the polypeptides to an animal, e.g., goat, rabbit, mouse, chicken, etc., preferably a non-human. The antibody so obtained will then bind the polypeptide itself. In this manner, even a sequence encoding only a fragment of the polypeptides can be used to generate antibodies binding the whole native polypeptides. Such antibodies can then be used to isolate the polypeptide from tissue expressing that polypeptide. Antibodies can also be generated by administering naked DNA. See, e.g., U.S. Pat. Nos. 5,703,055; 5,589,466; and 5,580,859.

For preparation of monoclonal antibodies, any technique, which provides antibodies produced by continuous cell line cultures can be used. Examples include, e.g., the hybridoma technique (Kohler and Milstein, Nature, 256:495-7 (1975)), the trioma technique, the human B-cell hybridoma technique (Kozbor et al., Immunology Today, 4:72 (1983)), and the EBV-hybridoma technique to produce human monoclonal antibodies (Cole et al., Monoclonal Antibodies and Cancer Therapy, Alan R. Liss, Inc., pp. 77-96 (1985)).

Techniques described for the production of single chain antibodies (e.g., U.S. Pat. No. 4,946,778) can be adapted to produce single chain antibodies to immunogenic polypeptide products of this invention. Also, transgenic animals may be used to express partially or fully humanized antibodies to immunogenic polypeptide products of this invention.

The invention also relates to other specific binding partners which include, e.g., aptamers and PNA.

Therapeutics

The methods of the present invention are also directed to facilitating the development of potentially useful therapeutic agents that may be effective in combating staphylococcal enterotoxin mediated or related diseases or conditions, and to methods of effecting such treatments.

The polypeptides, or variants or fragments thereof, of the invention can be administered to patients in need thereof by conventional procedures, in order to prevent or treat diseases or conditions as disclosed elsewhere herein and/or to ameliorate symptoms of those conditions. Such polypeptides can be formulated into pharmaceutical compositions comprising pharmaceutically acceptable excipients, carriers, etc., using conventional methodologies. Formulations and excipients, which enhance transfer (promote penetration) of an agent across the blood-brain barrier are also well known in the art.

In one embodiment, a therapeutically effective amount of a composition comprising a polypeptide of the invention is administered to a host in need thereof to block, prevent, or reduce the transcytosis of an SE thereby preventing or treating diseases or conditions as disclosed elsewhere herein and/or to ameliorate symptoms of those conditions.

In another embodiment, conventional methods of immunotherapy can be used. For example, therapeutically effective amounts of the polypeptides of the invention can be administered to a host as a vaccine to stimulate an immune response (i.e., to produce antibodies against the polypeptide-antigen)—but not cause disease—and thus providing protection against subsequent infection by an SE producing organism. Vaccines of the invention include preventive vaccines (e.g., measles or mumps) as well as therapeutic (treatment) vaccines. A vaccine of the invention can be administered through conventional methods (e.g., through needle injections, by mouth and by aerosol).

Antibodies can also be generated by administering naked DNA. See, e.g., U.S. Pat. Nos. 5,703,055; 5,589,466; and 5,580,859. Thus, another embodiment of the invention is a DNA vaccine comprising a polynucleotide of the invention.

In yet another embodiment, treatment methods according to the invention also encompass the administration of an antibody or fragment thereof to a patient in need of such therapy.

For any vaccine discussed above, it is well within the purview of the skilled artisan to determine dosage amounts as well as dose schedules for a particular host.

Transgenic Animals

The invention disclosed herein also relates to a transgenic animal comprising within its genome one or more copies of the polynucleotides encoding the novel polypeptides of the invention. The transgenic animals of the invention may contain within their genome multiple copies of the polynucleotides encoding the polypeptides of the invention, or one copy of a gene encoding such polypeptide but wherein said gene is linked to a promoter (e.g., a regulatable promoter) that will direct expression (preferably overexpression) of said polypeptide within some, or all, of the cells of said transgenic animal. In a preferred embodiment, expression of a polypeptide of the invention occurs preferentially in tissues of the digestive tract and/or lung tissue. A variety of transgenic organisms are encompassed by the invention, including e.g., drosophila, C. elegans, zebrafish and yeast. The transgenic animal of the invention is preferably a mammal, e.g., a cow, goat, sheep, rabbit, non-human primate, or rat, most preferably a mouse.

Methods of producing transgenic animals are well within the skill of those in the art, and include, e.g., homologous recombination, mutagenesis (e.g., ENU, Rathkolb et al., Exp. Physiol., 85(6):635-44, (2000)), and the tetracycline-regulated gene expression system (e.g., U.S. Pat. No. 6,242,667), and will not be described in detail herein. See e.g., Wu et al., Methods in Gene Biotechnology, CRC, pp. 339-366 (1997); Jacenko, O., Strategies in Generating Transgenic Animals, Recombinant Gene Expression Protocols, Vol. 62 of Methods in Molecular Biology, Humana Press, pp. 399-424 (1997).

Transgenic organisms are useful, e.g., for providing a source of a polynucleotide or polypeptide of the invention, or for identifying and/or characterizing agents that modulate expression and/or activity of such a polynucleotide or polypeptide. Transgenic animals are also useful as models for disease conditions related to, e.g., overexpression of a polynucleotide or polypeptide of the invention.

The present invention also relates to a transgenic non-human animal whose genome comprises one or more genes coding for a polypeptide disclosed herein. Most preferably said animal is a mouse.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-4 (1980); Palmiter et al., Cell, 41:343-5 (1985); Palmiter et al., Ann. Rev. Genet., 20:465-99 (1986); Askew et al., Mol. Cell. Bio., 13:4115-24 (1993); Games et al., Nature, 373:523-7 (1995); Valancius and Smithies, Mol. Cell. Bio., 11:1402-8 (1991); Stacey et al., Mol. Cell. Bio., 14:1009-16 (1994); Hasty et al., Nature, 350:243-6 (1995); Rubinstein et al., Nucl. Acid Res., 21:2613-7 (1993); Cibelli et al., Science, 280:1256-8 (1998). For guidance on recombinase excision systems, see, e.g., U.S. Pat. Nos. 5,626,159, 5,527,695, and 5,434,066. See also Orban, et al., “Tissue-and Site-Specific DNA Recombination in Transgenic Mice,” Proc. Natl. Acad. Sci. USA, 89:6861-5 (1992); O'Gorman, S., et al., “Recombinase-Mediated Gene Activation and Site-Specific Integration in Mammalian Cells,” Science, 251:1351-5 (1991); Sauer et al., “Cre-stimulated recombination at loxP-Containing DNA sequences placed into the mammalian genome,” Polynucleotides Research, 17(1):147-61 (1989); Gagneten et al., Nucl. Acids Res., 25:3326-31 (1997); Xiao and Weaver, Nucl. Acids Res., 25:2985-91 (1997); Agah et al., J. Clin. Invest., 100:169-79 (1997); Barlow et al., Nucl. Acids Res., 25:2543-5 (1997); Araki et al., Nucl. Acids Res., 25:868-72 (1997); Mortensen et al., Mol. Cell. Biol., 12:2391-5 (1992) (G418 escalation method); Lakhlani et al., Proc. Natl. Acad. Sci. USA, 94:9950-5 (1997) (“hit and run”); Westphal and Leder, Curr. Biol., 7:530-3 (1997) (transposon-generated “knock-out” and “knock-in”); Templeton et al., Gene Ther., 4:700-9 (1997) (methods for efficient gene targeting, allowing for a high frequency of homologous recombination events, e.g., without selectable markers); PCT International Publication WO 93/22443 (functionally-disrupted).

A polynucleotide according to the present invention can be introduced into any animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-5, (1985)), sheep (Hammer et al., Nature, 315:343-5, (1985)), cattle, rat, or primate. See also, e.g., Church, Trends in Biotech., 5:13-19 (1987); Clark et al., Trends in Biotech., 5:20-4, (1987)); and DePamphilis et al., BioTechniques, 6:662-80 (1988)). Transgenic animals can be produced by the methods described in U.S. Pat. No. 5,994,618, and utilized for any of the utilities described therein.

Computer-Based Applications

The nucleotide or amino acid sequences of the invention are also provided in a variety of media to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exist in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention, which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen on a three-dimensional configuration, which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBIA).

For example, software, which implements the BLAST (Altschul et al., J. Mol. Biol., 215:403-10 (1990)) and BLAZE (Brutlag et al., Comp. Chem., 17:203-7 (1993)) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1. SEB movement across epithelial cell monolayers. SEB (10, 50, 100 or 300 mg/ml) was added to the apical or basal side of confluent T-84 or Caco-2 cell monolayers. The opposing chamber was sampled over a 24 hour period, and the amount of toxin present determined by sandwich ELISA. Apical to basal movement is depicted as a solid line, and basal to apical movements is depicted as a dashed line. Each point represents the mean of three replicate experiments±standard error.

FIG. 2. Toxins (10-300 mg/ml) were added to the apical chamber of confluent T-84 monolayer inserts and incubated for 24 hours. The amount of toxin transcytosed to the basal chamber was determined by ELISA using only only HRP-conjugated antitoxin antisera (Toxin Technologies), and results are expressed as the mean of two replicate experiments±standard error. Significant differences between toxins were assessed by ANOVA followed by Scheffe's F test (p<0.05) and are indicated by a “*”.

FIG. 3. Peptide inhibition of SEB transcytosis. Peptides were incubated with 1 mg/ml SEB at the indicated ratios for 24 hours, and toxin transcytosis across T-84 cell monolayers was assessed by ELISA. Results from three replicates are presented as the mean SEB transcytosed±standard deviation. As indicated on the axis, in the absence of peptide, 46.1±13.4 ng/ml of SEB was transcytosed. SEB 61-92 at 1:10 toxin:peptide and SEB 151-180 at 1:10 and 1:100 were significantly different from toxin alone as determined by ANOVA followed by Scheffe's F-test (p<0.05).

FIGS. 4a and 4b. Molecular models of SEB, A, E and TSST-1. The amino acids highlighted in green (indicated by a dashed arrow) signify the conserved sequence (KKKVTAQELD in SEB). The yellow amino acids on SEB identify the areas implicated in MHC II binding and the red amino acids correspond to the binding site of the TcR. All structures were obtained from the Protein Data Bank of the Research Collaboratory for Structural Bioinformatics. Downloaded files were subsequently manipulated using the RasMol program. The coordinates are based upon original publications by: Papageorgiou, A. C., H. S. Tranter, K. R. Acharya. 1998. J Mol Bio. 277:61, Schad, E. M., I. Zaitseva, V. N. Vaitsev, M. Dohlsten, T. Kalland, P. M. Schlievert, D. H. Ohlendorf, L. A. Svensson. 1995. EMBO J. 14:3292. Swaminathan, S., W. Furey, J. Pletcher, M. Sax. 1995. Nat Struct Biol. 2:680, and Prasad, G. S., R. Radhakrishnan, D. T. Mitchell, C. A. Earhart, M. M Dinges, W. J. Cook, P. M. Schlievert, D. H. Ohlendorf. 1997. Protein Sci. 6:1220, for SEB, SEA, SEE, and TSST-1 respectively (FIG. 4a). Sequence conservation between SEB, SEA, SEE and TSST-1 (FIG. 4b).

FIG. 5. Inhibition of SEB transcytosis by SEB 152-161 and SEB 130-160. Peptides were incubated with 1 mg/ml SEB at the indicated ratios for 24 hours, and toxin transcytosis assessed. Results are presented as the mean±standard error SEB that was able to cross a monolayer of T-84 cells in the presence of the synthetic SEB peptides. In the absence of peptide, 46.1 ng/ml of SEB was transcytosed with a standard error of 9.6 ng/ml. Data was normalized based on transcytosis of toxin alone from three replicate experiments. SEB 130-160 at a 1:10 and 1:100 toxin:peptide ratio and SEB 152-161 at all ratios were significantly different from toxin alone as determined by ANOVA followed by Scheffe's F-test (p<0.05).

FIG. 6. Cross reactivity of anti-SEB peptide antisera with other SE. The reactivity of anti-SEB 152-161) and anti-SEB 130-160 to SEE, SEB, SEA and TSST-1 was assessed by ELISA. Data is represented as mean of four replicate determinations+/−standard deviation.

FIG. 7. Anti-peptide antisera inhibition of multipe toxin transcytosis. Antisera from mice immunized with SEB 152-161 or SEB 225-234 conjugated to KLH were incubated at a 1:100 dilution with 1 mg/ml SE or TSST-1, and toxin transcytosis assessed. Results are presented as the mean of two replicate experiments±standard error of SE or TSST-1 that was able to cross a monolayer of T-84 cells in the presence of anti-peptide antisera. The anti-SEB 152-161 significantly inhibited transcytosis of all four toxins as determined by ANOVA followed by Scheffe's F-test (p<0.05).

FIG. 8. SEB 152-161 Reduces Tissue Toxin Levels. Both toxin (50 μg) and peptide were administered i.p. SEB 152-161 inhibited the accumulation of SEB toxin in liver and kidney tissue.

FIG. 9. In vivo administration (both IP and oral) of SEB peptide results in antibody production

FIG. 10. Toxin-induced apoptosis of proximal kidney cells is reduced by peptide in vitro.

FIG. 11. This figure shows the reduction in toxin reaching tissues when co-administered with SEB peptide.

FIG. 12. SEB peptide reduced toxin induced lethality.

FIG. 13. Chronology of SEB-Induced Piglet Toxic Shock Syndrome. This graphic shows vital sign changes and gross pathology associated at different time points after SEB exposure. The stages compensated, decompensated, and irreversible shock are loosely assigned to show correlation with human disease.

FIG. 14. Vital Signs. A) Rectal body temperature measurements are shown in 12 hour intervals through 96 hours post exposure. Each data point represents the average measurement of 7-8 animals selected from at least 3 separate experiments ([SEB]=150 mg/kg and Control=equivalent volume of saline). B) Results of systolic blood pressure measurements, using Doppler sonography, are also shown in 12 hour increments through 96 hours post exposure. Each data point represents the mean of 7-8 animals from at least 3 separate experiments.

FIG. 15. SEB-Induced Gene Changes. PCR product was subjected to electrophoresis and bands were visualized using Cyber Green. Fluorescent intensity at each time was compared to that of saline control and data are presented as fold-change. Each point represents the mean of triplicate experiments. (Standard Deviations for 2, 6, 24, 48 and 72 hours, are: V1a—0.21, 0.29, 3.2, 0.16, and 4.8; sABP—0.5, 0.2, 0.2, 0.4, and 0.5; Egr1—0.07, 0.08, 0.22, 0.21, and 0.27; ATPase-alpha—0.22, 0.05, 0.30, 0.46, and 0.23; ATPase-beta—0.47, 0.99, 1.5, 2.53, and 1.8).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

Materials and Methods

Toxins, antitoxins and toxin peptides. Toxins, antitoxins, and antitoxin conjugates were purchased from Toxin Technology, Inc. (Sarasota, Fla.). SEA, B, E and TSST-1 were guaranteed 95 percent pure based on gel electrophoresis and subsequent Coomassie blue or silver staining. Based on the published sequence of SEB (Jones et al., J. Bacteriol., 166:29-33 (1986)) four 30 amino acid long synthetic peptides representing all but the amino and carboxy termini of the molecule were prepared by Peninsula Laboratories (Belmont, Calif.). SEB 152-161, SEB 130-160, and the two control peptides SEB 225-234 and SEB 191-220 from regions of the molecule not implicated in SEB function were synthesized with an ABI 431 A peptide synthesizer using Na-fluoroenylmethyloxycarbonyl chemistry. Peptides were cleaved from the resins using trifluoroacetic acid/ethanedithiol/thioanisole/anisole at a ratio of 90/3/5/2, extracted in ether and ethyl acetate and subsequently dissolved in water and lyophilized. Reverse phase HPLC analysis of crude peptides indicated one major peak in each profile, and amino acid analysis indicated actual composition corresponded closely to theoretical.

Anti-peptide antisera generation. A portion of each of the synthesized peptides was conjugated to keyhole limpet hemocyanin (KLH). 13 mg of peptide was conjugated to 9 mg of KLH using 200 mM glutaraldehyde, and the glutaraldehyde removed by dialysis. After dialysis the peptide-carrier solutions were diluted to a concentration of 5 mg/ml, sterile filtered and stored at −80° C. Ten week old C57BL/6 mice were purchased from NCI Charles River Laboratory (Frederick, Md.), and injected intraperitoneally with 250 μl of a 1:1 mixture of antigen (200 μg/ml) and Freund's complete adjuvant. On days 14 and 28, the mice received a boost of 250 μl of a 1:1 mixture of antigen (200 μg/ml) and Freund's incomplete adjuvant. Mice were euthanized on day 38, and blood was collected. Serum was removed and samples were assayed for IgG responses to SEB and other SE by enzyme-linked immunosorbent assay (ELISA) using toxin-coated plates. Titers were determined as the inverse of the dilution of serum that gave a half-maximal response. Control sera made against KLH alone did not react with toxins.

Cells. The human crypt-like colonic epithelial cell line, T-84, a model Cl⁻ secretory epithelium, and the human adenocarcinoma cell line, Caco-2 were chosen as a model epithelia. T-84 cells, especially, are used commonly in drug absorption modeling, and monolayers can achieve a predictable, high electrical resistance in culture. The stock cultures were obtained from American Type Culture Collection (Manassas, Va.). T-84 cells were maintained in equal parts DMEM and Ham's F-12 media supplemented with 10% fetal bovine serum (FBS), and 1.5% HEPES and used between passages 9-12. Cell suspensions (500 μl) at 5×10⁵ cells/ml were added to the chambers of 12-mm nitrocellulose filters (Millicell-HA; Millipore Corp., Bedford, Mass.) and placed into 24-well tissue culture plates containing 500 μl culture medium. Medium was replaced every other day until confluent monolayers with tight junctions developed as assessed by a hydrostatic pressure test and transepithelial electrical resistance (TEER) of monolayers. The hydrostatic pressure test was performed on filters 24 hours before an experiment. Each monolayer was filled with medium to the top and incubated overnight at 37° C. Any filter that showed gross examinable leakage was excluded from the trial. TEER across other membranes that passed the hydrostatic pressure test was measured with a Millicell-ERS instrument (Millipore Corp.). Only monolayers with TEER of ≧150 Ω×cm² after correction for intrinsic value of empty filter were used. At this point the medium in both the filter inserts (apical medium) and culture wells (basal medium) were replaced, and the cultures were used in the transcytosis modeling protocol.

Caco-2 cells were maintained in Earle's Minimal Essential Medium (with L-glutamine) which was supplemented with 20% FBS, 1 mM sodium pyruvate, and 0.1 mM non-essential amino acids. Caco-2 cell monolayers were prepared using the Biocoat® HTS Caco-2 Assay System (Becton Dickinson, Franklin Lakes N.J.). Each system contains a fibrillar collagen-coated 24-well plate. Cells were seeded 4×10⁵ and supplemented with “Basal Seeding Medium.” After 24 hours media was replaced with “MITO+ Serum Extender.” On day three the monolayers were tested for confluence (as described above). The HTS system was employed because of its three-day turn-around time.

Toxin transcytosis assay. The ability of T-84 or Caco-2 cells to transcytose SE and TSST-1 was tested using confluent monolayers grown on filter inserts as described above. Media was removed from the selected filter area (apical or basal), and 500 μl of fresh culture medium (without FBS) containing various concentrations of toxin was then added. Another 500 μl of culture medium (without FBS) was added to the other filter area. After incubation at 37° C. for the indicated period of time, apical or basal media were collected depending on where the toxins were added. Inhibition assays were conducted similarly, and potential inhibitors (peptides or anti-peptide antisera) were added to the wells that contained toxins at appropriate concentrations.

ELISAs. Nunc-Immuno™ MaxiSorp™ plates were coated with 100 μl of a 0.01 mM carbonate buffer solution containing 10 μg/ml of anti-toxin IgG. Coated plates were placed in a humid chamber over night on a plate rocker at 37° C. After incubation, plates were washed 3 times with PBS containing 2.5% Tween and then refilled a fourth time with PBS/Tween and allowed to incubate for 15 minutes, as recommended by Toxin Technologies for detection of low toxin concentrations. Samples (50 μl) were then added to the wells of the plate in triplicate. A standard curve was created using known concentrations of the toxin. Plates were incubated in humidified chamber for 2 hours at 37° C. and subsequently washed 3 times. 100 μl of horseradish peroxidase conjugated anti-toxin was then added to all wells and incubated as described above for 1 hour. Plates were washed 5 times followed by addition of o-phenylenediamine dihydrochloride substrate. After color development, the reaction was stopped with 2 M sulfuric acid. Plates were then read on a microplate reader at a wavelength of 490 nm. Titers of antitoxin antisera were determined using standard ELISA procedure and anti-immunoglobulin antibodies (Sigma).

### EXAMPLE I

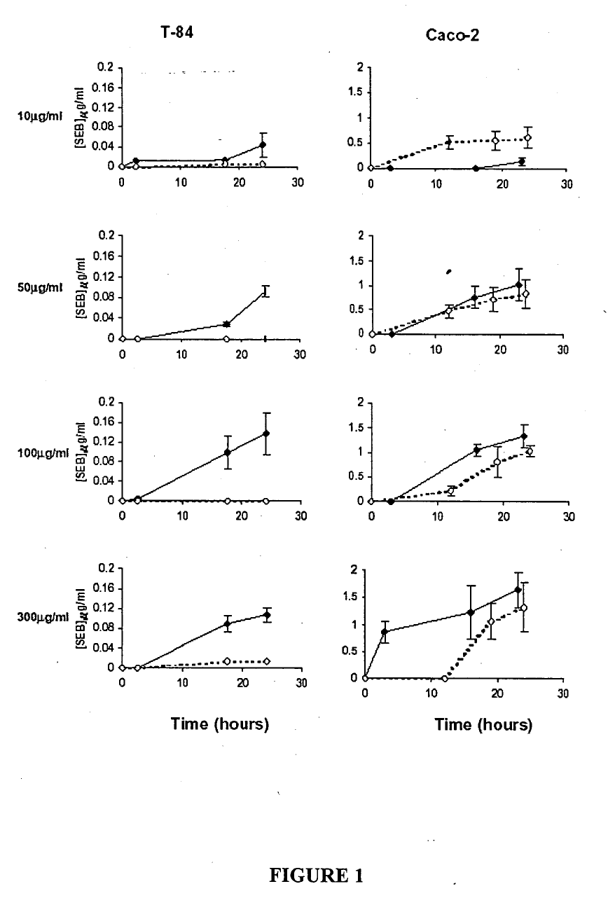

Toxin transcytosis across epithelial monolayers. The movement of SEB across monolayers of T-84 and Caco-2 cells was determined over a 24 hour period (FIG. 1). In both cells lines, SEB transcytosis increased with both increasing dose and time, but a higher percentage of administered toxin was transcytosed at lower concentrations. SEB transcytosis has been previously suggested to be a receptor-facilitated process (Hamad et al., J. Exp. Med., 185:1447-1454 (1997)), and in T-84 but not Caco-2 cells, saturation was observed at the 100. μg/ml toxin dose. In T-84 cells, transcytosis was biased in the apical to basal direction, which is anticipated in a polarized cell line like T-84 with unequal receptor distribution. Although there was little polarization observed in Caco-2 monolayers, this is consistent with previous observations and the demonstration of bi-directional receptors in this cell line (Ellis et al., J. Biol. Chem., 270:20717-23 (1995); Hamad et al., J. Exp. Med., 185:1447-54 (1997)). Given the polarization of the T-84 monolayers, receptor saturation, and the consistent production of monolayers with high electrical resistance, T-84 cells were used for further experiments.

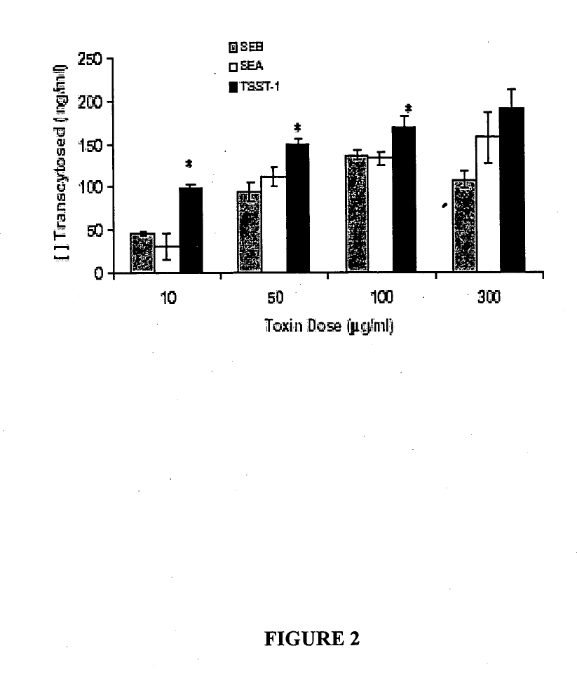

Movement of SEB was contrasted with that of SEA and TSST-1 (FIG. 2). SEA apical to basal transcytosis across T-84 monolayers was roughly parallel to the movement of SEB. It was only significantly higher at the 300 μg/ml concentration. Transcytosis of TSST-1 was slightly greater than that of SEB at all doses examined.

### EXAMPLE II

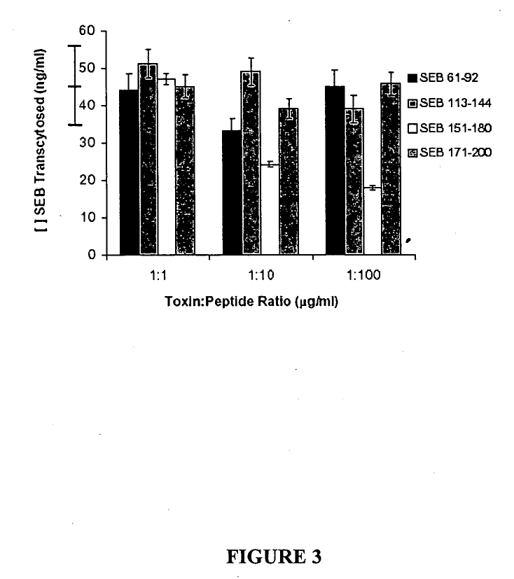

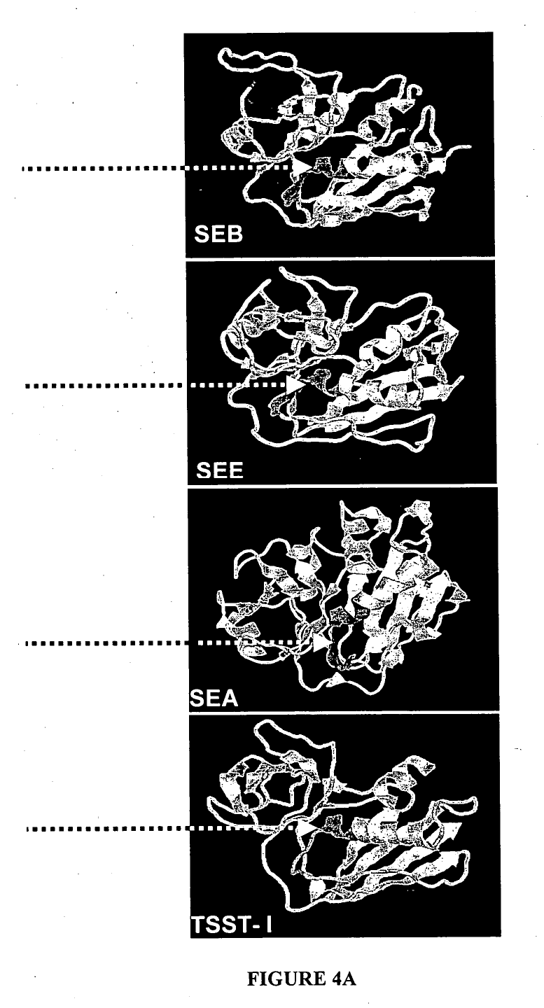

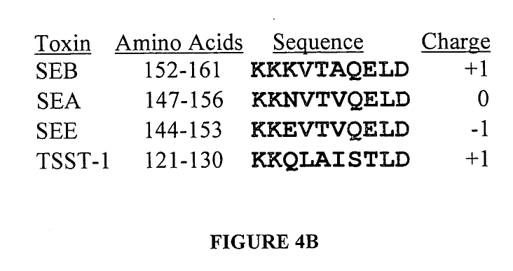

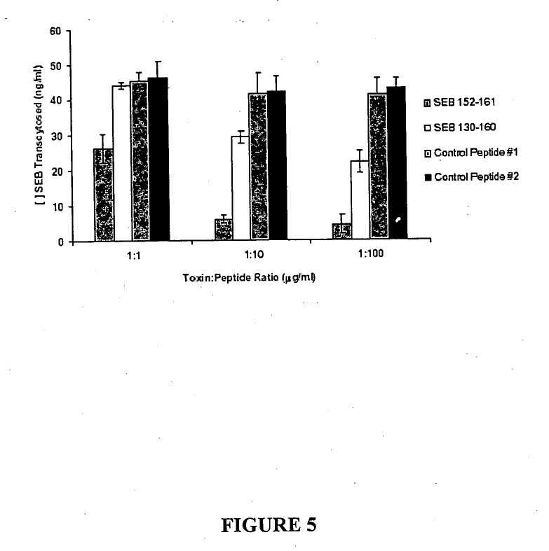

A conserved SEB peptide blocks SEB transcytosis. Synthetic peptides corresponding to various regions of SEB were used to attempt to block SEB transcytosis (FIG. 3). The peptides employed encompassed all but the amino and carboxy terminal regions. Only one peptide of the four examined, SEB 151-180, inhibited SEB movement across T-84 monolayers in a dose-dependent manner. Sequence analysis of the region corresponding to this peptide indicated that a 10 amino acid sequence from residue 152 to 161 of SEB was highly conserved among other SE and some conservation was even seen with TSST-1 (FIGS. 4a and 4b). Further, structural similarity of this 10 amino acid region was observed among SE. In SEB, this region includes the end of the twisted beta sheet 7, beta sheet 8—also twisted, and the beginning of helix 4 (Swaminathan et al., Nature, 359:801-806 (1992)), and this conformation is retained in other SE and TSST-1. Therefore, the conserved 10 amino acid peptide, SEB 152-161, was synthesized. A longer peptide, SEB 130-160, with an extension into beta sheet 6 was also produced in an attempt to maintain the protruding position of valine 155, since longer peptides have been shown to better retain native structures. In addition, D161 was omitted to increase the overall charge of the peptide to +2, overcoming solubility problems. SEB 152-161 and SEB 130-160 along with control peptides of similar sizes from regions of SEB not previously identified asfunctionally significant were used to inhibit SEB transcytosis (FIG. 5). Both peptides containing the conserved sequence exhibited dose dependent inhibition of SEB movement across T-84 cell monolayers. The control peptides were without effect. Unexpectedly, the conserved 10 mer was a more potent competitor than the longer peptide since even at a 1:1 ratio with toxin, significant inhibition of transcytosis was observed. Thus, the conserved sequence consisting of residues 152-161 of SEB appears to represent a region of the molecule involved in transcytosis of toxin across epithelial cell monolayers.

### EXAMPLE III

SEB 152-161 inhibits transcytosis of multiple SE. SEB 152-161 was examined for its ability to affect transcytosis of SEA, SEE and TSST-1 (Table 1). Consistent with the peptide inhibition of SEB transcytosis, SEB 152-161 inhibited movement across epithelial cell monolayers of all three other SE examined in a dose dependent manner. Transcytosis of the more highly homologous SEA and SEE was reduced 73% and 68%, respectively. TSST-1 was also sensitive to inhibition with the SEB peptide, since at a toxin to peptide ratio of 1:100 its transcytosis was reduced by 59%.

TABLE I
SEB Peptide Inhibition of Transcytosis of Other SEs*

| Toxin:Peptide | | | |
|---|---|---|---|
| Ratio | SEA | SEE | TSST-1 |
| Toxin Alone | 60 ± 4.0 | 43 ± 12 | 31 |
| 1:1 | 46.9 ± 2.2 | 44.7 ± 0.5 | 35.2 ± 3.7 |
| 1:10 | 20.5 ± 0.9† | 19.1 ± 0.5† | 18.5 ± 1.8† |
| 1:100 | 15.9 ± 0.8† | 13.7 ± 2.3† | 12.7 ± 4.1† |

*Peptides were incubated with 1 μg/ml SE at the indicated ratios for 24 hours, and toxin transcytosis assessed by ELISA. Results are expressed as the mean of three replicate determinations ± standard deviation except for TSST-1. Significant differences from toxin controls were assessed by ANOVA followed by Scheffe's F test (p <0.05) and are indicated by a “†”.

### EXAMPLE IV

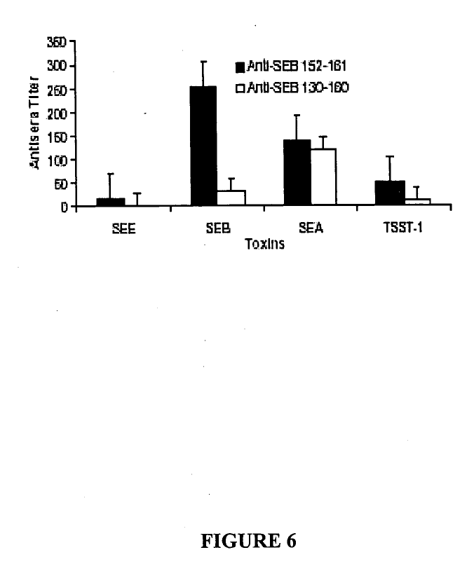

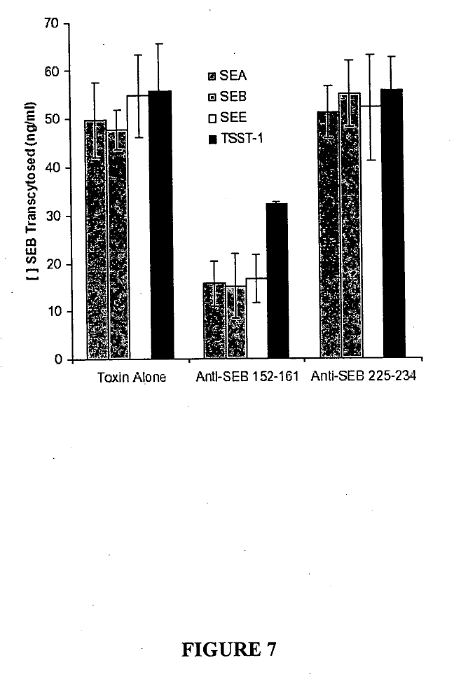

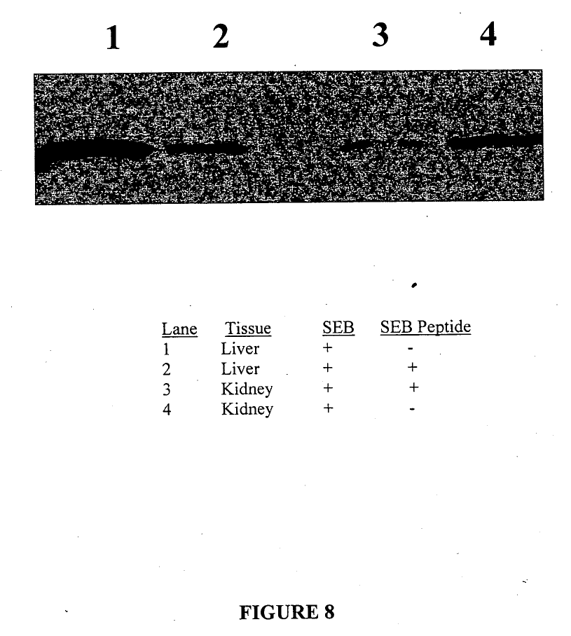

Antisera against the conserved peptide inhibits transcytosis of SEB. To confirm the region of the molecule encompassing SEB 152-161 as important for SEB transcytosis, antipeptide antisera was generated in mice and used to inhibit SEB movement. Antisera to the 10-mer peptide displayed greater reactivity with the parent toxin, SEB than did antisera to the larger SEB 130-160 (FIG. 6). Cross reactivity of the antipeptide antisera with other SE was also observed, especially with SEA which could be anticipated based on the extent of homology. In contrast, little cross reactivity to SEE was seen which could be a function of the change in charge at from a K in SEB to an E in SEE. Anti SEB 152-161 was used in the SEB transcytosis assay because of its greater reactivity with SEB. Anti-SEB 152-161 significantly reduced transcytosis of not only SEB, but SEA, SEE, and TSST-1 as well while control antisera was without effect (FIG. 7). Therefore, the toxin peptide was antigenic and the antipeptide antisera inhibited movement of all SE examined.

### EXAMPLE V

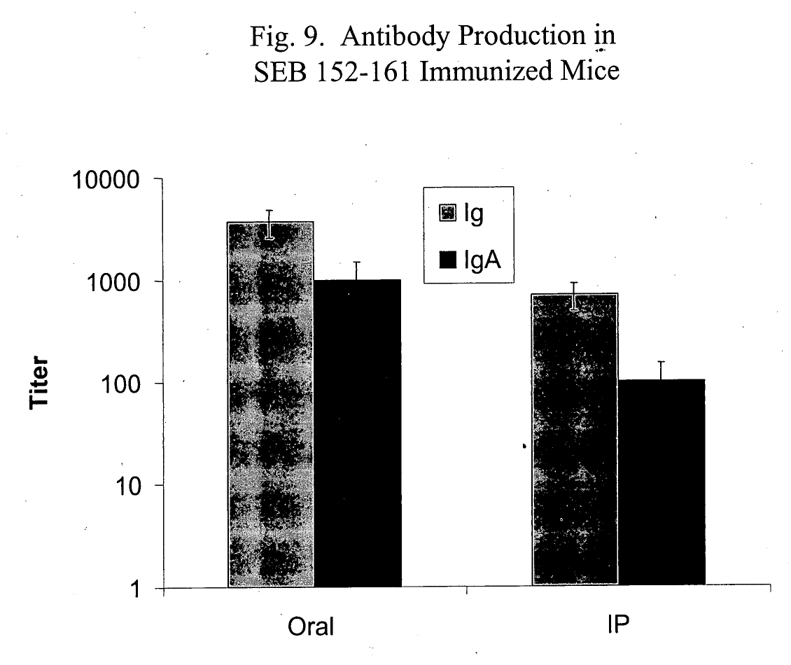

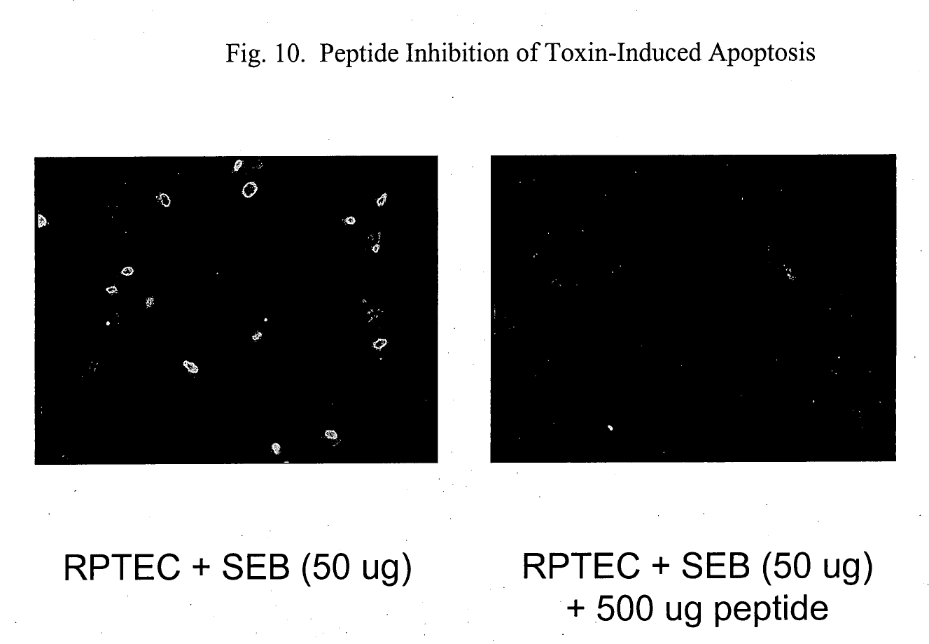

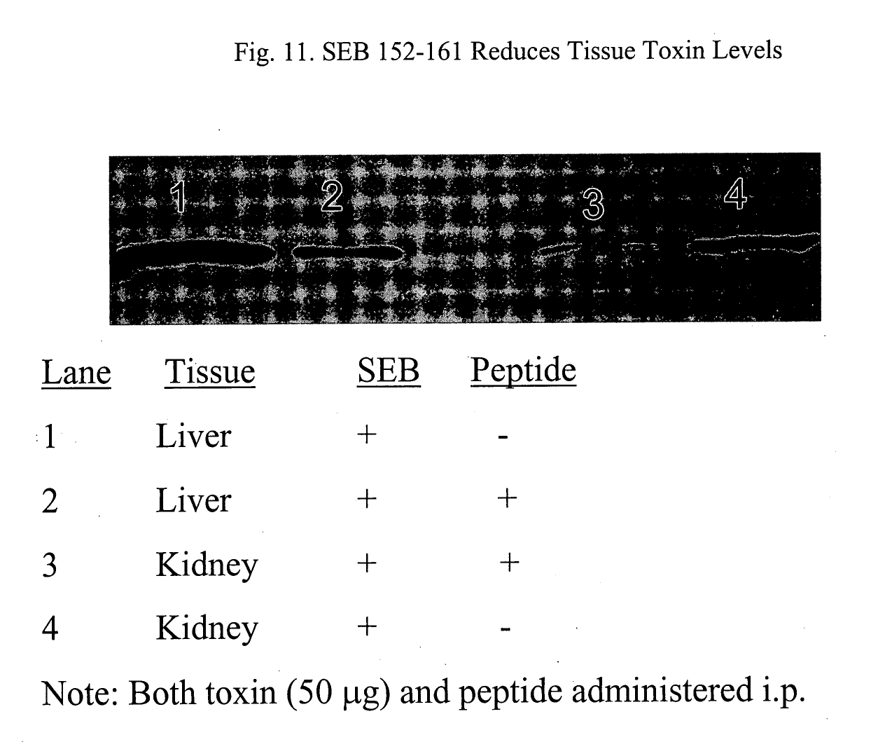

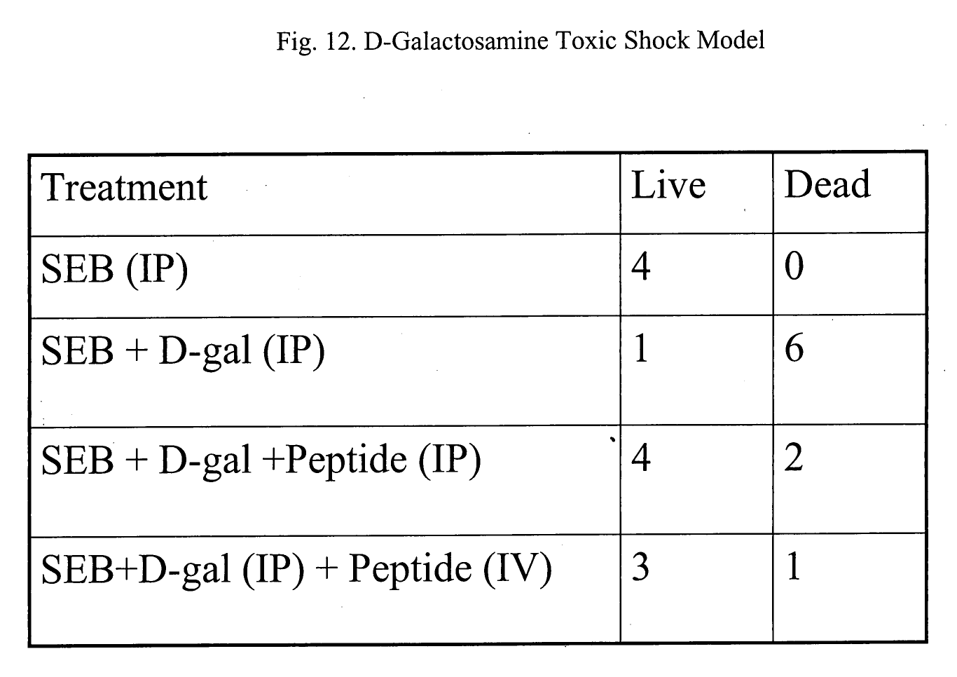

As shown in FIG. 9, in vivo administration of SEB peptide to mice elicited antibody protection. This protection was accompanied by a reduction in toxin-induced apoptosis of proximal kidney cells (FIG. 10). SEB polypeptide reduced the amount of toxin reaching cells when co-administered with toxin (FIG. 11). Finally, it was shown SEB peptide reduced toxin induced lethality in a whole animal toxic shock model (FIG. 12).

### EXAMPLE VI

Materials and Methods

Animals

Litters of ˜8, 12-day-old, male and female Yorkshire piglets were obtained from Archer Farms (Darlington, Md.) and housed in groups of ˜3 piglets (assigned by treatment) in metal runs (˜3′×10′) lined by rubber mats. Piglets were maintained under controlled lighting (12-hour light-dark cycle), at a temperature of 85° F. and humidity of ˜60%. Animals were fed swine pre-starter complete feed (Hubbard Feeds, Mankato, Minn.). Piglets had continual access to feed, water and a 2-3 heat lamp sources at one end of the run. At ˜18-days of age, anesethetized piglets (isofluorane (3% initially, achieving maintenance at ˜1.5-2%)) (Abbott Labs, North Chicago, Ill.) received a lethal dose of SEB (150 mg/kg) or an equivalent volume of saline, administered into the ear vein using a 26 g 3/4 inch catheter. At 4, 6, 24, 48, 72 or 96 hours post treatment, animals were anesthetized with isofluorane, terminal measurements and blood were obtained and the piglets were euthanized using Buthanasia-D (Burns Biotech, Omaha, NB) administered via intracardiac injection.

Toxin Preparation

SEB, lot 14-30, purified by the method of Schantz et al (22), was stored as a dry powder in pre-measured vacuum ampules. A working stock solution was made by dissolving the SEB in sterile pyrogen-free water to achieve a concentration of 5 mg/ml and that solution was aliquoted and stored frozen. At the time of use, an appropriate aliquot was thawed and diluted with IV injectable saline to 300 mg/ml. LD˜95 was achieved using 150 mg/kg.

Clinical Observations and Measurements

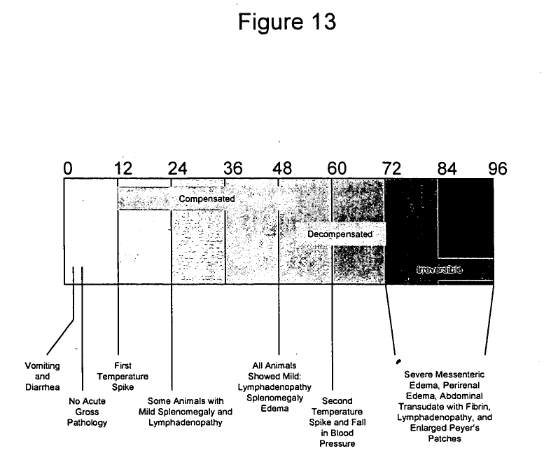

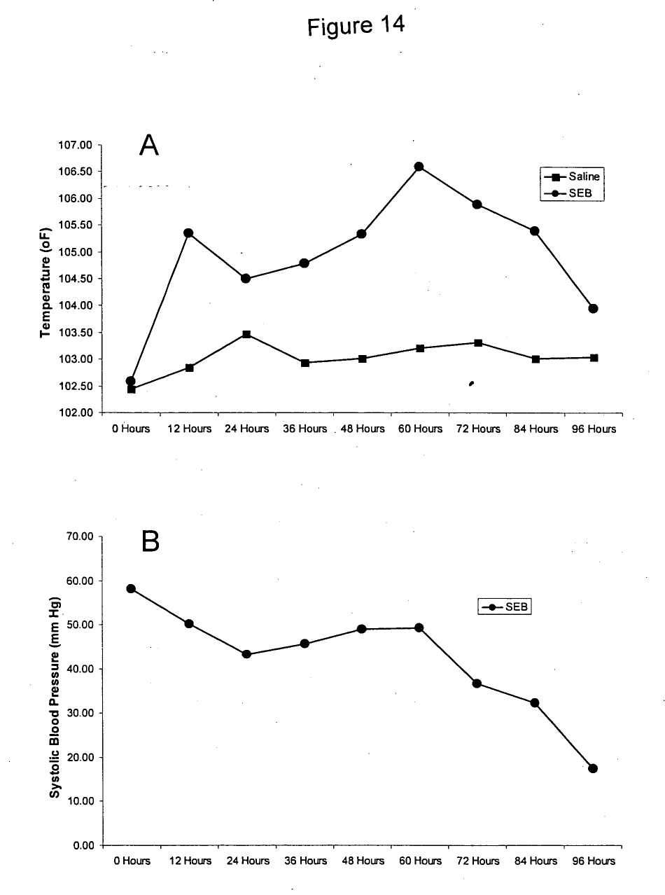

Animals were monitored continuously for clinical signs for the first 18 hours post treatment and every 6 hours until euthanasia. Recorded clinical observations included piglet symptoms for at least 3 piglets per time period and for 3 different experiments (FIG. 13). Rectal body temperature was measured at least hourly 0-12 h and 1-2× daily thereafter (FIG. 14a). Systolic blood pressure was measured by Ultrasonic Doppler Flow Detector (Model 811BL; Parks, Medical Electronics; Aloha, Oreg.) (FIG. 14b).

Gross and Microscopic Pathology

After euthanasia a complete necropsy was performed as follows: 4 hours (1 piglet), 6 hours (1 piglet), 24 hours (5 piglets), 48 hours (5 piglets), 72 hours (7 piglets) and 96 hours (4 piglets). At least one saline control piglet was examined per litter, with a total of 7 saline controls. A full set of tissues from each animal was fixed in 10% neutral buffered formalin. Fixed tissues were routinely trimmed, embedded in paraffin, sectioned at 5-7 mm and stained with hematoxylin and eosin for microscopic examination. Tissues examined microscopically for this report were: thymus, stomach, jejunum, spiral colon, descending colon, liver, spleen, pancreas, kidney, adrenal gland, urinary bladder, multiple lymph nodes, lung, heart, and brain.

Gene Studies

Whole blood samples were collected into CPT™ Vacutainer™ tubes (BD, Franklin Lakes, N.J.) at various time points and processed in accordance with the manufacturer's specifications which allow for the enrichment of peripheral mononuclear cells (PBMC). Total RNA was subsequently isolated from PBMCs using TRIzol reagent (Life Technologies, Grand Island, N.Y.) following manufacturer protocol.

Preliminary gene array yielded data that implicated several gene profile changes post-SEB treatment (data not presented). Five representative genes were chosen and primer pairs to be used for PCR were designed based on known mRNA sequences (Genbank, PubMed) using Primer3 software or Genelooper 2.0 from Geneharbor.

Equal amounts of total RNA were reversed transcribed to cDNA using oligo (dT)12-18 and Superscript reverse transcriptase II (Invitrogen, Carlsbad, Calif.). The obtained cDNA was used as a template for PCR reactions using PCR master mixture (Roche, Indianapolis, Ind.). Each cDNA was subjected to 25-30 PCR cycles using a GeneAmp 9600 thermal cycler (Perkin Elmer, Norwalk, Conn.) with conditions that resulted in a single specific amplification product of the correct size. Amplification was empirically determined to be in the linear range. mRNA amounts were normalized relative to 18S rRNA. Reaction products (10 ml) were visualized after electrophoresis on a 1% agarose gel using SYBR Green I (Kemtek, Rockville, Md.). Gels were digitized using a BioRad Molecular Imager FX (BioRad, Hercules, Calif.) and band intensities were used to calculate mRNA abundance.

Results

Clinical Signs

Administration of IV SEB at 150 mg/kg was lethal (or deemed non-survivable by the attendant veterinarian) in 31/31 piglets. This concentration of toxin was found most effective after preliminary dose/response experiments using a range of 30-200 μg/kg. After administration of the SEB, pre-established behavioral characteristics were recorded for each animal as a function of time post exposure during the course of the experiment (continually for the first 6 h and intermittently during the rest of the experiment). Five descriptions of piglet behavior for each of 3 categories (healthy, incapacitation, prostration) were established based on observed behavior from other studies with piglets. The animals showed onset of typical incapacitation signs (transient vomiting [˜3-6 episodes], severe diarrhea, anorexia) that began at 0.8-1.5 h post exposure (FIG. 13). The diarrhea, anorexia persisted during the remainder of the experiment. From 3-7 h, the animals stayed huddled together in a remote area of the cage, showing continually increasing signs of prostration. Once an animal progressed to a state of unrecoverable shock (as determined by attendant veterinarian) euthanasia was provided.

Plotted rectal temperatures showed two febrile peaks at 12 and 60 hours with the 60 h time point being most extreme. Around day 3 temperatures began to fall and showed no evidence of homeostatic recovery (FIG. 14a). Systolic blood pressures were variable throughout most of the time course however a distinct hypotensive trend was observed at or around the third day of observation (FIG. 14b).

Gross Findings

Gross changes were progressive over time. No significant gross changes were present in the piglets necropsied at 4 and 6 hours post SEB treatment or in any saline control animals. By 24 hours mildly enlarged mesenteric lymph nodes and mild splenomegaly were present in 2 of 5 animals. By 48 hours post SEB treatment all animals had consistent mild splenomegaly when compared to control animals and diffuse mild to moderate enlargement of the mesenteric lymph nodes that were often bright to dark red. Six of seven animals at this time point had mild to moderate perirenal, mesenteric, gall bladder and gastric wall edema and mildly enlarged and congested peripheral lymph nodes. Two of the seven animals had prominent red peyer's patches and a marked abdominal transudate with strings of fibrin.

Gross lesions were most remarkable at 72 and 96 hours. All animals necropsied at these time periods had severe mesenteric edema that was most prominent between loops of spiral colon, as well as perirenal edema, variable edema of the gall bladder and gastric wall and mild diffuse subcutaneous edema. This was accompanied by a marked abdominal transudate (protein, 2.5 g/dL, hypocellular) with strands of fibrin. Mesenteric lymph nodes were greatly enlarged, dark red and sometimes contained multifocal white areas of necrosis. Peripheral lymph node involvement was similar and varied from minimal to severe. Peyer's patches were often prominent and red (congested).

Microscopic Findings

Histologic examination of selected tissues confirmed gross observations and helped to further characterize changes. The general progression of histologic changes in the mesenteric lymph nodes was: mild lymphoid hyperplasia by 24 hours, progression to moderate lymphoid hyperplasia and congestion by 48 hours, and marked lymphoid necrosis with hemorrhage, edema and fibrin accumulation by 72 to 96 hours. Mild to moderate diffuse lymphoid hyperplasia was present in mesenteric lymph nodes in all animals examined at 24 hours post exposure. At 48 hours, all mesenteric lymph nodes examined had moderate to severe diffuse lymphoid hyperplasia. Many blood vessels in these nodes were congested and the loose peripheral tissue analogous to medullary sinuses contained many free erythrocytes. In addition, there were few small scattered areas of hemorrhage and lymphoid necrosis. Lymphoid necrosis was much more extensive in 6 of 7 and 3 of 3 mesenteric lymph nodes examined at 72 and 96 hours respectively. At these time points extensive lymphoid necrosis characterized by abundant karryorhectic debris was accompanied by marked hemorrhage and edema often with fibrin lining small caliber vessels and prominent fibrin thrombi. Changes in the peripheral lymph nodes were similar but much less severe and tended to occur at the later time periods.

Lymphoid hyperplasia was also present in all spleens examined at 24 hours post treatment and later. This change was characterized by mild diffuse expansion of the periarteriolar lymphoid sheaths (PALS). The lymphocytes in the affected PALS were larger, with increased cytoplasm and a large irregularly round stippled nucleus and there were increased numbers of mitotic figures in these areas.

Severe mesenteric edema between loops of spiral colon seen grossly at 2 and 96 hours was verified histologically. Microscopically mesenteric connective tissue was loosely arranged and widely separated by a lightly eosinophilic to clear material and delicate eosinophilic fibrillar material (edema) and many extravasated red blood cells. Mesenteric lymphatics were consistently ectatitc.

Additional histologic findings included lymphoblastic perivascular infiltrates and mild portal lymphoplasmacytic hepatitis. Small perivascular lymphocytic cuffs were present in the lungs of most animals examined at 48 hours and later (5 of 6 and 48 hours, 7 of 7 at 72 hours and 3 of 4 at 96 hours) and in the brain of two animals examined at 96 hours. Cuffs often contained evidence of lymphoid necrosis with accumulation of karyorrhectic debris. Mild lymphoplasmacytic portal hepatitis was variably present at 24 hours and later: 3 of 5 piglets at 24 hours, 3 of 5 piglets at 48 hours, 6 of 7 piglets at 72 hours and 1 of 4 piglets at 96 hours.

SEB-Induced Gene Changes

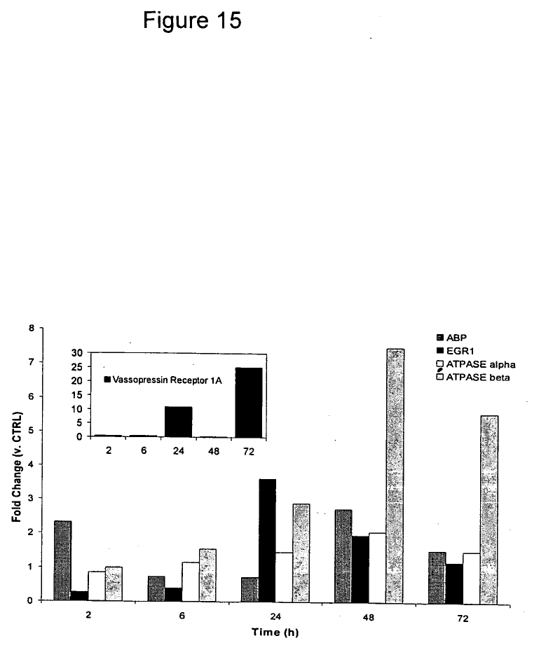

After initial survey using custom gene microarrays, five genes were selected for study at 2, 6, 24, 48, and 72 hours post SEB exposure using RT-PCR (FIG. 15). mRNA levels for vasopressin receptor 1a (V1a), a peripheral receptor associated with vasoconstriction, were markedly increased at 24 and 72 hours (˜10-fold and ˜25-fold respectively). Interestingly the timing of the V1a gene changes coincide with observed systolic blood pressure changes graphed in FIG. 2a. Na, K-ATPase subunits a and b gene profiles showed a time dependent increase which were greatest at 48 hours. Although both subunits followed a similar trend, the b isoform proved to have a larger increase as compared to that of the a isoform (˜8-fold, v. ˜2-fold at 48 hours). Early growth response gene 1 (Egr1), a key transcription factor implicated in many disease processes including hypoxia, showed an increase at all time points. Most remarkably was an increase in mRNA levels at the 24-hour time point. Finally, the gene profile for the soluble angiotensin binding protein (sABP) was also increased at all time points with highest levels found at 48 hours.

Discussion

We have developed a clinically relevant piglet model of lethal SEB intoxication that is superior to the current monkey and rodent models. This model more realistically parallels SEB intoxication in people than described mouse models and piglets are easier to obtain, maintain and handle than the non-human primate model.

This piglet model exhibits a biphasic clinical response to SEB intoxication that is virtually identical to that described in people but is not described in mouse models. Although lethal SEB intoxication has been achieved in previously manipulated mouse models, none of these models exhibit the typical initial gastrointestinal signs described in humans. In addition, the small size of these animals makes obtaining many clinical measurements such as repeat routine hematology, serum chemistries, blood pressure and body temperature difficult.

The monkey model of lethal SEB intoxication is clinically superior to mouse models. Interestingly, the subtle clinical biphasic response to SEB intoxication shown by rhesus monkeys is not as exuberant or easily detected and monitored as that seen in this piglet model. This is likely a result of the fact that the laboratory Rhesus monkey retains many behavioral characteristics of its wild counterpart, including remarkable masking of clinical disease, which increases survival under natural adverse conditions; this is in marked contrast to the domestic pig whose disposition has been markedly altered by selective breeding. In addition, working with non-human primates, especially rhesus macaques, comes with a unique set of limitations, most notably high expense, limited supply and biosafety concerns. The aggressive nature of these monkeys and complications associated with Herpes B positive colonies make heavy sedation or anesthesia necessary for many routine procedures. In contrast, the piglets used in this model are easy to obtain and relatively inexpensive. The social nature of these animals allows routine procedures to be performed without anesthesia or sedation and with minimal stress to the animal and handler.

In addition, study of other porcine models of human disease indicate that this species shows strong similarities to humans with respect to vascular responsiveness (23) and is a good model in which to study cardiovascular disease. In fact, Lee et al (24) used porcine aortic endothelial cells to demonstrate that TSST-1, has a direct toxic effect on endothelium. There is also a described swine model of septic shock that culminates in a hypotensive crisis (25) that is similar to that observed in this model.

We have shown that administration of intravenous SEB to piglets results in terminal hypotension and shock similar to that seen in toxic shock syndrome in people and SEB intoxication in the rhesus macaque. Postmortem findings in people, monkeys and piglets indicate that hypotension and shock in SEB intoxication is a result of leakage of fluid from vessels into extravascular spaces. Pulmonary edema is the most consistent and remarkable gross lesion associated with death in the primate model of intravascular SEB intoxication (16, 26) and in people with toxic shock syndrome (27). One major difference in this piglet model compared to the disease in humans is that terminal edema is predominantly focused on the abdomen rather than the thorax resulting in severe mesenteric and perirenal edema with comparatively minor edema at other sites. It is interesting to note, that other natural and experimental angiotoxic diseases in the pig result in vascular leakage with edema predominantly in the abdominal region. In edema disease, a well characterized porcine disease, direct endothelial binding of Shiga-like toxin type IIe (SLT-IIe) secreted by E. coli, results in marked spiral colon mesenteric edema similar to that seen in this SEB piglet model (28). In another porcine model that displays classical signs of circulatory shock, edema of the gastric wall and gall bladder is a result of experimental intravenous administration of T-2 toxin, a mycotoxin secreted by Fusarium species thought to cause moldy corn disease in swine (29). The abdominally focused edema in pigs may constitute a species difference that should be considered, especially in research aimed at treating late stage hypotensive shock and pulmonary edema. However, we feel that this is still a valid model for lethal SEB pathogenesis studies and treatment trials.

Another characteristic unique to swine is the unique porcine lymph node architecture. Porcine lymph nodes are essentially reversed from other mammalian lymph nodes in that lymphoid tissue is centrally located and surrounded by loose peripheral lymphoreticular tissue resembling the medullary sinuses in other species. Although porcine lymph nodes are morphologically different, the functional flow of lymph is essentially identical to other species (30) and in the author's (YAV) opinion does not represent a significant species difference.

Histological lesions in this piglet model are similar to those described in other animal models of SEB intoxication. Ulrich et al (17) provides a detailed description of both pulmonary and non-pulmonary lesions associated with lethal aerosol SEB exposure in the rhesus macaque. This model also had wide spread T-lymphocyte hyperplasia with enlarged lymph nodes, expanded PALS and circulating lymphoblasts. In addition, lymphocytic portal infiltrates similar to those seen in this model were also reported in the exposed monkeys. Another report of lethal aerosol SEB exposed monkeys described pulmonary perivascular lymphocytic infiltrates similar to those seen in this study (31). Lymphoid hyperplasia followed by lympholysis in the spleen is described in an Actinomycin-D primed mouse model (21). A similar change was noted in a mouse model of aerosol SEB exposure (Vogel, P.A., personal communication). These findings are consistent with the immunological manifestations of SAg exposure.

As in the mouse models marked lympholysis was apparent in most piglets at 72 and 96 hours post SEB administration. However, this change was limited to severely affected lymph nodes and was not apparent in the thymus or spleen. It is possible that the severe lymphoid depletion noted at autopsy of several lethal cases of human toxic shock syndrome (27) was a sequela of massive lympholysis. As TSS is lethal only in a small percentage of cases (27) it is interesting to hypothesize that this change may be associated with lethality.

The febrile state of treated animals is of particular interest and raises many questions. Studies using SEA mutants suggest that the emetic and superantigenic activity of SEs may be separate (32). Immediately following exposure, piglets presented with an emetic phase that was not associated with temperature increase. Marked temperature elevation was not recorded in animals until after the last emetic event. If superantigenic T cell stimulation and subsequent cytokine production was solely responsible, one would suspect that the timing of emesis and fever would closely overlap. These data support the previously discerned hypothesis that the gastrointestinal and pyrogenic effects of SE may in fact be of different mechanism.

The timing of clinical symptoms, vital measurements, and pathologic lesions appears to be in direct concert (FIG. 13). The initial phases of intoxication caused severe incapacitation, and occurred in the absence of gross or histological pathology. Animals appeared to recover after initial onset, left only with residual diarrhea and fever. Gross lesions appear to develop around 24 hours corresponding with a further increase in body temperature. At hour 60 animal temperatures began to fall, corresponding with incremental reductions in systolic blood pressure and marked progression of pathologic lesions.

In order to elucidate pathways responsible for SEB lesions we have begun to profile gene changes. In this study we present data on five genes that were flagged subsequent to preliminary gene array surveying (FIG. 15). The transcription factor Egr1, is shown to have altered expression in the face of hypoxia (33, 34). Also, Egr family members have been implicated in the non-lymphoid expression of FasL and TNF (35). Interestingly, mRNA levels of Egr1 were highest at 24 h when the first signs of pathological lesions became evident. sABP, a binding protein for angiotensin is found widely distributed in peripheral tissues and also the brain (36). Although its physiologic relevance is uncertain, sABP may play a role in the balance of smooth muscle contraction (37). V1a, unlike V2a, is recognized to initiate vasoconstriction upon binding of its ligand vasopressin (anti-diuretic hormone, ADH). This vasoconstriction is part of a large compensatory response that mobilizes in instances of hypotension. By increasing peripheral vasculature resistance, blood pressure can be returned to a level that ensures adequate tissue perfusion. In this study, V1a mRNA levels are increased notably at 24 h, a time when systolic blood pressure re-equilibrates, and these levels are further increased at 72 h in beginning of hypotensive crisis.

The complex nature of SE pathophysiology has posed many questions scientifically and much of the host's response to these toxins has been explained through immunology. As we progress further in understanding the chronology and severity of lesions induced by SEB, it will be necessary to further investigate SEs interaction with non-immunological tissue. Most notably would be the correlation of SEs effect on endo and epithelia and presence of irreversible shock.

In summary we have characterized the clinical syndrome and post mortem findings of a 14-day-old Yorkshire piglet model of lethal SEB intoxication. This model is superior to previously described models.

### REFERENCES

1. Jett M, Brinkley W, Neill R, Gemski P, Hunt R. Staphylococcus aureus enterotoxin B challenge of monkeys: correlation of plasma levels of arachidonic acid cascade products with occurrence of illness. Infect Immun 58 (11): 3494-9, 1990.
2. Jarraud S, Peyrat M A, Lim A, Tristan A, Bes M, Mougel C, Etienne J, Vandenesch F, Bonneville M, Lina G. egc, a highly prevalent operon of enterotoxin gene, forms a putative nursery of superantigens in Staphylococcus aureus. J Immunol 166 (1): 669-77, 2001.
3. Marrack P, Kappler J. The staphylococcal enterotoxins and their relatives [published erratum appears in Science 1990 Jun. 1; 248(4959):1066] [see comments]. Science 248 (4956): 705-11, 1990.
4. Miethke T, Wahl, C., Heeg, K., Echtenacher, B., Krammer, P., and Wagner, H. T cell-mediated lethal shock triggered in mice by the superantigen staphylococcal enterotoxin B: critical role of tumor necrosis factor. Journal of Experimental Medicine 175 (1): 91-98, 1992.
5. Johnson H M, Torres B A, Soos J M. Superantigens: structure and relevance to human disease. Proc Soc Exp Biol Med 212 (2): 99-109, 1996.
6. Jardetzky T S, Brown J H, Gorga J C, Stem L J, Urban R G, Chi Y I, Stauffacher C, Strominger J L, Wiley D C. Three-dimensional structure of a human class II histocompatibility molecule complexed with superantigen. Nature 368 (6473): 711-8, 1994.
7. Johnson H M, Russell J K, Pontzer C H. Staphylococcal enterotoxin microbial superantigens. Faseb J 5 (12): 2706-12, 1991.
8. Hamad A R, Marrack P, Kappler J W. Transcytosis of staphylococcal superantigen toxins. J Exp Med 185 (8): 1447-54, 1997.
9. Shupp J W, Jett M, Pontzer C H. Identification of a transcytosis epitope on staphylococcal enterotoxins. Infect Immun 70 (4): 2178-86, 2002.
10. McKay D M, Singh P K. Superantigen activation of immune cells evokes epithelial (T84) transport and barrier abnormalities via IFN-gamma and TNF alpha: inhibition of increased permeability, but not diminished secretory responses by TGF-beta2. J Immunol 159 (5): 2382-90, 1997.
11. Chatterjee S, Khullar, M., and Shi, W. Y. Digalactosylceramide is the receptor for staphylococcal enterotoxin-B in human kidney proximal tubular cells. Glycobiology 5 (3): 327-333, 1995.
12. Chatterjee S, Jett M. Glycosphingolipids: the putative receptor for Staphylococcus aureus enterotoxin-B in human kidney proximal tubular cells. Mol Cell Biochem 113 (1): 25-31, 1992.
13. Normann S J. Renal fate of staphylococcal enterotoxin B. Lab Invest 25 (2): 126-32, 1971.
14. Campbell W N, Fitzpatrick M, Ding X, Jett M, Gemski P, Goldblum S E. SEB is cytotoxic and alters EC barrier function through protein tyrosine phosphorylation in vitro. Am J Physiol 273 (1 Pt 1): L31-9, 1997.
15. Normann S J, Jaeger R F, Johnsey R T. Pathology of experimental enterotoxemia. The in vivo localization of staphylococcal enterotoxin B. Lab Invest 20 (1): 17-25, 1969.
16. Stiles J W, Denniston J C. Response of the Rhesus monkey, Mucaca mulatta, to continuously infuded Staphylococcal Entertoxin B. Lab Invest 25 (6): 617-625, 1971.
17. Ulrich R G, Sidell S, Taylor T J, Wilhelmsen C L, Franz D R. Staphylococcal enterotoxin B and related pyrogenic toxins. In: Sidell F R, Takafuji E T, Franz D R, eds. Textbook of Military medicine. Part I Warfare, Weaponry, and the Casualty. Medical Aspects of Chemical and Biological Warefare. Washington: Office of the Surgeon General, pp. 621-630, 1997.
18. Savransky V, Rostapshov V, Pinelis D, Polotsky Y, Korolev S, Komisar J, Fegeding K. Murine lethal toxic shock caused by intranasal administration of staphylococcal enterotoxin B. Toxicol Pathol 31 (4): 373-8, 2003.
19. Anderson M R, Tary-Lehmann M. Staphylococcal enterotoxin-B-induced lethal shock in mice is T-cell-dependent, but disease susceptibility is defined by the non-T-cell compartment. Clin Immunol 98 (1): 85-94, 2001.
20. Yeung R S, Penninger J M, Kundig T, Khoo W, Ohashi P S, Kroemer G, Mak T W. Human CD4 and human major histocompatibility complex class II (DQ6) transgenic mice: supersensitivity to superantigen-induced septic shock. Eur J Immunol 26 (5): 1074-82, 1996.
21. Chen J Y, Qiao Y, Komisar J L, Baze W B, Hsu I C, Tseng J. Increased susceptibility to staphylococcal enterotoxin B intoxication in mice primed with actinomycin D. Infect Immun 62 (10): 4626-31, 1994.
22. Schantz E J, Roessler W G, Wagman J, Spero L, Dunnery D A, Bergdoll M S. Purification of staphylococcal enterotoxin B. Biochemistry 4 (6): 1011-6, 1965.
23. Feletou M, Teisseire B. Vascular pharmacology of the micropig: Importance of the endothelium. In: Swindle M M, Moody D C, Phillips L D, eds. Swine as Models in Biomedical Research. Ames: Iowa State Universtiy Press, pp. 74-95, 1992.
24. Lee P K, Vercellotti G M, Deringer J R, Schlievert P M. Effects of staphylococcal toxic shock syndrome toxin 1 on aortic endothelial cells. J Infect Dis 164 (4): 711-9, 1991.
25. Hoban L D, Paschall J A, Eckstein J, Nadkari V, Lee C R, Williams T J, Reusch D, Nevola J J, Carcillo J A. Awake porcine model of intrperitoneal sepsis. In: Swindle M M, Moody D C, Phillips L D, eds. Swine as Models in Biomedical Research. Ames: Iowa State Universtiy Press, pp. 246-264, 1992.
26. Finegold M J. Interstitial pulmonary edema. An electron microscopic study of the pathology of Staphylococcal enterotoxemia in Rhesus monkeys. Lab Invest 16 (6): 912-924, 1967.
27. Larkin S M, Williams D N, Osterholm M T, Tofte R W, Posalaky Z. Toxic shock syndrome: clinical, laboratory, and pathologic findings in nine fatal cases. Ann Intern Med 96 (6 Pt 2): 858-64, 1982.
28. Gelberg H B. Alimentary system. In: McGavin M D, Carltom W W, Zachary J F, eds. Thomson's Special Veterinary Pathology. 3 ed. St. Louis: Mosby, pp. 42-43, 2001.
29. Pang V F, Lorenzana R M, Beasley V R, Buck W B, Haschek W M. Experimental T-2 toxicosis in swine. III. Morphologic changes following intravascular administration of T-2 toxin. Fundam Appl Toxicol 8 (3): 298-309, 1987.
30. Landsverk T. Immune system. In: Dellmann D, Eurell J A, eds. Textbook of Veterinary Histology. 5 ed. Baltimore: Williams & Wilkins, pp. 137-142, 1998.
31. Mattix M E, Hunt R E, Wilhelmsen C L, Johnson A J, Baze W B. Aerosolized staphylococcal enterotoxin B-induced pulmonary lesions in rhesus monkeys (Macaca mulatta). Toxicol Pathol 23 (3): 262-8, 1995.
32. Hoffman M, Tremaine M, Mansfield J, Betley M. Biochemical and mutational analysis of the histidine residues of staphylococcal enterotoxin A. Infect Immun 64 (3): 885-90, 1996.
33. Jin K, Mao X O, Eshoo M W, del Rio G, Rao R, Chen D, Simon R P, Greenberg D A. cDNA microarray analysis of changes in gene expression induced by neuronal hypoxia in vitro. Neurochem Res 27 (10): 1105-12, 2002.
34. Ten V S, Pinsky D J. Endothelial response to hypoxia: physiologic adaptation and pathologic dysfunction. Curr Opin Crit Care 8 (3): 242-50, 2002.
35. Droin N M, Pinkoski M J, Dejardin E, Green D R. Egr family members regulate nonlymphoid expression of Fas ligand, TRAIL, and tumor necrosis factor during immune responses. Mol Cell Biol 23 (21): 7638-47, 2003.
36. Hagiwara H, Sugiura N, Wakita K, Hirose S. Purification and characterization of angiotensin-binding protein from porcine liver cytosolic fraction. Eur J Biochem 185 (2): 405-10, 1989.
37. Webb R C, Kiron M A, Rosenberg E, Soffer R L. Antiserum to angiotensin-binding protein inhibits vascular responses to angiotensin II. Am J Physiol 255 (6 Pt 2): H1542-4, 1988.

The entire disclosures of all applications, patents and publications, cited herein are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

| | |
|---|---|
| SEQ ID NO:1 = | |
| KKKVTAQELD | (SEB 152-161 amino acid) |
| SEQ ID NO:2 = | |
| RSITVRVFEDGKNLLSFDVQTNKKKVTAQEL | (SEB 130-160 amino acid) |
| SEQ ID NO:3 = | |
| KKNVTVQELD | (SEA 147-156 amino acid) |
| SEQ ID NO:4 = | |
| KKEVTVQELD | (SEE 144-153 amino acid) |
| SEQ ID NO:5 = | |
| KKQLAISTLD | (TSST-1 121-130 amino acid) |
| SEQ ID NO:6 = | |
| NKKKVTAQELDYLTRHYLVKNKKLYEFNNS | (SEB 151-180 amino acid) |
| SEQ ID NO:7 = | |
| AAGAAAAAGGTGACTGCTCAAGAATTAGAT | (SEB 152-161 nt) |
| SEQ ID NO:8 = | |
| AGAAGTATTACTGTTCGGGTATTTGAAGATGGTAAAAATTTATTATC | (SEB 130-160 nt) |
| TTTTGACGTACAAACTAATAAGAAAAAGGTGACTGCTCAAGAATTA | |
| SEQ ID NO:9 = | |
| ATTAAGAAAAAGGTGACTGCTCAAGAATTAGATTACCTAACTCGTCA | (SEB 151-180 nt) |
| CTATTTGGTGAAAAATAAAAAACTCTATGAATTTAACAACTCG | |
| SEQ ID NO:10 = | |
| AAGAAAAATGTAACTGTTCAGGAGTTGGAT | (SEA 147-156 nt) |
| SEQ ID NO:11 = | |
| AAAAAAGAAGTAACTGTTCAAGAGCTAGAT | (SEE 144-153 nt) |
| SEQ ID NO:12 = | |
| AAAAAACAATTAGCTATATCAACTTTAGAC | (TSST-1 121-130 nt) |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding International Patent application No. PCT/US03/03139, filed Feb. 3, 2003, and U.S. Provisional Application Ser. No. 60/353,365, filed Feb. 1, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. An isolated polypeptide comprising a sequence of SEQ ID NO:1, SEQ ID NO:2, SEQ ID NO:3, SEQ ID NO:4, SEQ ID NO:5, SEQ ID NO:6 or a fragment thereof.

2. The polypeptide of claim 1, wherein said polypeptide is SEQ ID NO:1, SEQ ID NO:2, SEQ ID NO:3, SEQ ID NO:4, SEQ ID NO:5 or SEQ ID NO:6.

3. The polypeptide of claim 2, wherein said fragment is at least 10 amino acids in length.

4. An isolated polypeptide comprising the sequence of SEQ ID NO:1.

5. A composition comprising a polypeptide of SEQ ID NO:1 and a pharmaceutically acceptable carrier.

6. A composition comprising a polypeptide of claim 1 and a pharmaceutically acceptable carrier.

7. A composition comprising a polypeptide of claim 2 and a pharmaceutically acceptable carrier.

8. A method of preventing a staphylococcal enterotoxin-mediated disease or condition comprising administering a therapeutically effective amount of a polypeptide of claim 1.

9. The method of claim 8 wherein said staphylococcal enterotoxin-mediated disease or condition is emesis, diarrhea, toxic shock, or immunosuppression.

10. A method of reducing transcytosis of a staphylococcal enterotoxin comprising administering a therapeutically effective amount of a polypeptide of claim 1.

11. The method of claim 10 wherein said staphylococcal enterotoxin is staphylococcal enterotoxin A-J or TSST-1.

12. A method of treating a staphylococcal enterotoxin-mediated disease or condition comprising administering a therapeutically effective amount of a polypeptide of claim 1.

13. A method of reducing systemic exposure to a staphylococcal enterotoxin comprising administering a therapeutically effective amount of a polypeptide of claim 1.

14. An antibody that binds to a polypeptide of claim 1.

15. A vaccine comprising a polypeptide of claim 1.

16. A method of preventing a staphylococcal enterotoxin-mediated disease or condition comprising administering the antibody of claim 14.

17. The method of claim 16 wherein said staphylococcal enterotoxin-mediated disease or condition is emesis, diarrhea, toxic shock, or immunosuppression.

18. A method of reducing transcytosis of a staphylococcal enterotoxin comprising administering a therapeutically effective amount of the antibody of claim 14.

19. The method of claim 18 wherein said staphylococcal enterotoxin is staphylococcal enterotoxin A-J or TSST-1.

20. A method of treating a staphylococcal enterotoxin-mediated disease or condition comprising administering a therapeutically effective amount of the antibody of claim 14.

21. A method of reducing systemic exposure to a staphylococcal enterotoxin comprising administering a therapeutically effective amount of the antibody of claim 14.

22. A method of preventing a staphylococcal enterotoxin-mediated disease or condition comprising administering a therapeutically effective amount of the vaccine claim 15.

23. The method of claim 22 wherein said staphylococcal enterotoxin-mediated disease or condition is emesis, diarrhea, toxic shock, or immunosuppression.

24. A method of reducing transcytosis of a staphylococcal enterotoxin comprising administering a therapeutically effective amount of the vaccine of claim 15.

25. The method of claim 24 wherein said staphylococcal enterotoxin is staphylococcal enterotoxin A-J or TSST-1.

26. A method of treating a staphylococcal enterotoxin-mediated disease or condition comprising administering a therapeutically effective amount of the vaccine of claim 15.

27. A method of reducing systemic exposure to a staphylococcal enterotoxin comprising administering a therapeutically effective amount of the vaccine of claim 15.

28. An isolated polynucleotide comprising a sequence of SEQ ID NO:7, SEQ ID NO:8, SEQ ID NO:9, SEQ ID NO:10, SEQ ID NO:11, SEQ ID NO:12 or a fragment thereof.

29. The polynucleotide of claim 28 wherein said polynucleotide is SEQ ID NO:7, SEQ ID NO:8, SEQ ID NO:9, SEQ ID NO:10, SEQ ID NO:11, SEQ ID NO:12.

30. An isolated polynucleotide which encodes a polypeptide of SEQ ID NOS:1-6 or a fragment of a polypeptide of SEQ ID NOS:1-6.

31. An isolated polynucleotide comprising the sequence of SEQ ID NO:7.

32. An isolated polypeptide that is at least 40% homologous to a polypeptide of claim 1 and inhibits transcytosis of a staphylococcal enterotoxin.

33. The isolated polypeptide of claim 32, wherein said polypeptide is at least 70% homologous.

34. An isolated polynucleotide that is at least 40% homologous to a polynucleotide of claim 28 and encodes a polypeptide that inhibits transcytosis of a staphylococcal enterotoxin.

35. The isolated polynucleotide of claim 34, wherein said polynucleotide is at least 70% homologous.

36. A composition comprising a polynucleotide of SEQ ID NO:7 and a pharmaceutically acceptable carrier.

37. A composition comprising a polynucleotide of claim 28 and a pharmaceutically acceptable carrier.

38. A composition comprising a polynucleotide of claim 29 and a pharmaceutically acceptable carrier.

39. A composition comprising the antibody of claim 14 and a pharmaceutically acceptable carrier.

40. A vaccine comprising the polypeptide of claim 2.

41. A vaccine comprising the polynucleotide of claim 28.

42. A vaccine comprising the polynucleotide of claim 29.

43. A recombinant construct comprising a polynucleotide of claim 28.

44. A recombinant construct comprising a polynucleotide of claim 29.

45. A cell comprising the construct of claim 43.

46. A cell comprising the construct of claim 44.

47. A method for making the cell of claim 45 comprising introducing into said cell a construct comprising a polynucleotide of SEQ ID NOS:7-12.

48. A method for making a polypeptide of SEQ ID NOS:1-6, comprising incubating the cell of claim 47 under conditions in which the polypeptide is expressed and harvesting the polypeptide.

49. A transgenic animal comprising at least one polynucleotide of claim 28.

50. A transgenic animal comprising at least one polynucleotide of claim 29.

51. An antibody that binds to a polypeptide of claim 2.

52. A method of preventing a staphylococcal enterotoxin-mediated disease or condition comprising administering a therapeutically effective amount of the vaccine claim 41.

53. A method of reducing transcytosis of a staphylococcal enterotoxin comprising administering a therapeutically effective amount of the vaccine of claim 41.

54. A method of treating a staphylococcal enterotoxin-mediated disease or condition comprising administering a therapeutically effective amount of the vaccine of claim 41.

55. A method of reducing systemic exposure to a staphylococcal enterotoxin comprising administering a therapeutically effective amount of the vaccine of claim 41.

56. A composition for oral or inhaled administration which is for treating and/or preventing a staphylococcal enterotoxin-mediated disease or condition, comprising effective amounts of SEQ, ID NO:1, SEQ ID NO:2, SEQ ID NO:3, SEQ ID NO:4, SEQ ID NO:5, SEQ ID NO:6 or a fragment thereof, wherein said amounts are effective for directly inhibiting SEB transcytosis.

57. A composition of claim 56, wherein said composition dos not evoke an immune response.

58. A method of testing agents for treating and/or preventing a staphylococcal enterotoxin-mediated disease or condition, comprising
administering an agent for treating and/or preventing a staphylococcal enterotoxin-mediated disease or condition to a piglet, wherein the piglet has been administered an amount of SEB which is effective for causing a staphylococcal enterotoxin-mediated disease or condition, and
determining whether said agent is effective for treating and/or preventing a staphylococcal enterotoxin-mediated disease or condition.
